(19) United States
(12) Patent Application Publication
(10) Pub. No.: US 2017/0022242 A1
(43) Pub. Date: Jan. 26, 2017

(54) NOVEL ANTIVIRAL AND ANTITUMORAL COMPOUNDS

(71) Applicant: KATHOLIEKE UNIVERSITEIT LEUVEN, KU LEUVEN R&D, Leuven (BE)

(72) Inventors: Piet Herdewyn, Heverlee (BE); Munmum Maiti, Leuven (BE); Ling-Jie Gao, Bierbeek (BE); Steven De Jonghe, Tervuren (BE)

(73) Assignee: KATHOLIEKE UNIVERSITEIT LEUVEN, KU LEUVEN R&D, Leuven (BE)

(21) Appl. No.: 15/302,223

(22) PCT Filed: Apr. 17, 2015
(86) PCT No.: PCT/EP2015/058431

§ 371 Date: Oct. 6, 2016

(30) Foreign Application Priority Data
Apr. 17, 2014 (GB) 1406927.2
Jan. 20, 2015 (GB) 1500897.2

(51) Int. Cl. C07H 19/06; A61K 31/7072; A61K 31/7068

(52) U.S. Cl. CPC C07H 19/06; A61K 31/7068; A61K 31/7072

(57) ABSTRACT

The present invention relates to novel phosphate-modified nucleosides, such as phosphoramidate nucleosides. The invention also relates to the use of these novel phosphate-modified nucleosides to treat or prevent viral infections and proliferative diseases (such as cancer) and their use to manufacture a medicine to treat or prevent viral infections and proliferative diseases particularly infections with viruses belonging to the HCV family.

### FIELD OF THE INVENTION

The present invention relates to novel phosphate-modified nucleosides, such as phosphoramidate nucleosides. The invention also relates to the use of these novel phosphate-modified nucleosides to treat or prevent viral infections and proliferative diseases (such as cancer), and their use to manufacture a medicine to treat or prevent viral infections and proliferative diseases particularly infections with viruses belonging to the HCV family.

### BACKGROUND OF THE INVENTION

Nucleosides and nucleotides have demonstrated wide-spread utility as antiviral agents. All antiviral nucleosides are essentially prodrugs since their antiviral activity depends upon their intracellular metabolism within virus-infected cells to form sequentially the mono-, di- and triphosphates. It is these nucleotides, and especially the triphosphates that are the pharmacologically active species, as they are incorporated into a growing DNA or RNA strand by a DNA or RNA polymerase, resulting in chain termination or fraudulent DNA/RNA. The first phosphorylation step leading to the formation of the nucleoside 5′-monophosphate is commonly catalyzed by a nucleoside kinase encoded by the host cell or the virus infecting the host cell. Conversion of the nucleoside monophosphate to the corresponding 5′-diphosphate and triphosphates is carried out by nucleoside, nucleotide, and nucleoside diphosphate kinases, respectively. Hence, cellular kinases, as well as virally-encoded kinases play a vital role in the activation of nucleoside drugs.

In addition, nucleosides are also well known for their antitumoral activity. The mechanism of action for most of these compounds is very similar. They are intracellularly converted to their respective nucleotide analogues, which inhibit DNA synthesis by inhibition of DNA polymerase (as the nucleoside-triphosphate) and/or ribonucleotide reductase (as the corresponding nucleoside diphosphate).

In most cases, the first step of phosphorylation represents the rate-limiting step of the bioactivation and its inefficiency may limit the therapeutic potential of the nucleoside analogues. It has for example been documented that long-term administration of AZT leads to a decreased activity of thymidine kinase (which is the first phosphorylating enzyme) and thus resistance. This type of resistance is observed, not only in host tissues of patients receiving AZT, but also in viruses. Another example includes the antiviral compound acyclovir, which activity against HSV is dependent on the specific phosphorylation of the compound by the viral encoded thymidine kinase.

Bypassing this rate-limiting activation step may improve the biological activity of the nucleosides. In principle, administration of nucleoside-5′-monophosphates would overcome the drawbacks. However, phosphates are strongly acidic, and thus negatively charged at physiological pH and hence, are not able to penetrate the lipid-rich cell membrane. In addition, phosphohydrolases (acid and alkaline phosphatases, 5′-nucleotidases) rapidly convert the phosphates to the corresponding nucleosides.

In order to overcome to poor cellular permeability of nucleoside 5′-monophosphates, it has been proposed by Montgomery that ‘this difficulty might be overcome if one could prepare an ester of a nucleotide which could penetrate the cell wall and then be metabolized to the nucleotide itself’. Consequently, various prodrug or ‘pronucleotide’ approaches have been devised and investigated. In general, the goal of these approaches has been to promote stability in the extracellular medium, passive diffusion through cell membranes and to liberate the parent nucleotide intracellulary, where it can be further phosphorylated to the pharmacologically active species.

Several prodrug approaches now exist which have been recently reviewed by Wagner et al. (Med. Res. Rev. 2000, 20, 417-451). Examples include the cyclosal approach, discovered by Meier and co-workers. This is a class of prodrugs that depend mainly on chemical hydrolysis for activation, in which the nucleoside analogue monophosphate and salicyl alcohol is intracellularly released.

HepDirect Prodrugs are designed to undergo an oxidative cleavage reaction catalyzed by cytochrome P450 isoenzymes, expressed predominantly in the liver. This type of prodrug is useful for the delivery of phosphate or phosphonate containing drugs into the liver.

Neutral lipophilic alkyl and aryl phosphotriesters are very cell-permeable. However, in general, because of their stability, conversion to the corresponding triphosphate is problematic. Therefore, a number of biolabile protecting moieties have been described. For example, the SATE (S-Acyl-2-thioethyl) approach. Their activation is initiated by the carboxyesterase-mediated hydrolysis of the thioester moiety of one of the SATE groups to form the unstable O-2-mercaptoethylphosphotriester. The thiol generated, which is a soft nucleophile, attacks the soft electrophilic methylene carbon atom, releasing ethylene sulfide and the monoSATE phosphodiester which is then most likely subjected to hydrolysis by an intracellular phosphodiesterase to generate nucleoside monophosphate and S-acyl-thioethanol or a second carboxyesterase mediated thioester hydrolysis generates nucleoside monophosphate. Very similar to the SATE approach is the Dithioethyl (DTE) approach, that takes advantage of the greater reducing potential within the cells to liberate the nucleotide intracellularly. As a result of the reductase-mediated reductive cleavage, the unstable O-2-mercaptoethyl monoDTE phosphotriester is formed, followed by release of ethylene sulfide or thioethanol by an intramolecular nucleophilic displacement. The monoDTE phosphodiester can undergo hydrolysis mediated by phosphodiesterase or undergo a second reductase-mediated disulfide cleavage to generate nucleotide.

Another class of pro-nucleotides are the bis(pivaloyloxymethyl)-[POM] phosphotriesters. This approach utilizes a carboxyesterase-catalyzed cleavage of the pivaloyl ester within the POM-masking group to yield the highly reactive O-2-hydroxymethyl phosphotriester which spontaneously eliminates formaldehyde to give the monoPOM phosphodiester. The carboxyesterase which is used for this activation process is thought to be more prevalent inside the cells. To obtain the free nucleotide, this enzymatic activation has to be repeated or, alternatively, a phosphodiesterase cleaves the phosphodiester directly to yield the nucleotide. An analogues approach is the bis(isopropyloxycarbonyloxymethyl) [bis(POC)] nucleotide. This modification uses a carbonate diester as the masking group. The degradation pathway is similar to bis(POM)-nucleotide metabolism. In contrast to the bis(POM)-approach, the bis(POC) modification avoids the formation of two equivalents of pivalic acid that accumulate in the cells and potentially cause toxicity.

The aryl phosphoramidate class of prodrugs has been developed by McGuigan et al. in early 1990s. The cleavage of this class of prodrug is initiated by esterase enzyme, then an intramolecular cyclization is believed to take place with displacement of the aryl moiety to form a short-lived five-membered ring intermediate, which is hydrolyzed to phosphoramidic acid. The cleavage of the monoamidate to the active species may be catalyzed by a second enzyme like phosphoramidase or may result from simple hydrolysis in a more acidic subcellular compartment, releasing intracellularly nucleoside-monophosphate.

Several successful examples of ProTide analogues have been reported in literature. For example, the ProTide chemistry has been applied to gemcitabine, a well-known anticancer agent. It was concluded that L-alanine based phosphoramidates were optimal for antitumoral activity (J. Med. Chem. 2014, 57, 1531-1542) and one of the congeners is currently being evaluated in phase I/II clinical trials for its antitumoral activity.

Sofosbuvir is a uridine nucleotide prodrug and now marketed under the trade name Sovaldi® with rapid intestinal absorption and is easily taken up by hepatocytes from the circulation. Intracellularly, the side chains on the phosphate are removed and the 2′-deoxy-2′-fluoro-2′-C-methyluridine monophosphate GS-606965 is converted into the pharmacologically active uridine triphosphate. After binding of the nucleotide to the RNA chain, further addition of nucleotides is not possible and chain elongation is terminated. The drug was approved by the FDA in December 2013 for the treatment of HCV genotypes 2 and 3 in combination with ribavirin, and for genotypes 1 and 4 in combination with pegylated IFN and ribavirin.

Besides phosphoramidates, the phosphono amidate prodrug strategy has also been applied. An example includes GS-7340, which is the phenyl monoester isopropyl alaninyl phosphono amidate of the anti-HIV drug Tenofovir.

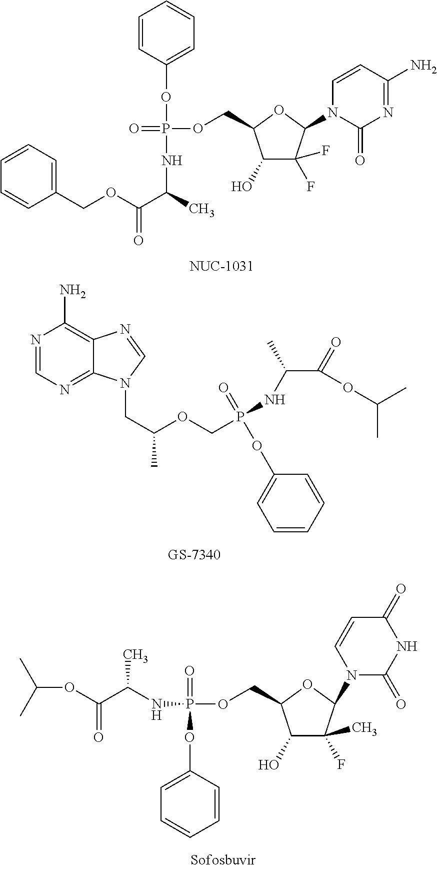

The prior art, especially the McGuigan prior art as mentioned hereabove, teaches the use of L-alanine as the preferred amino acid motif in the aryloxyphosphoramidate ProTides prodrug design. Other amino acids, such as L-aspartic acid di-ester (more particularly C₁ or C₂-esters) have been evaluated for antiviral activity and were found to be less active than L-alanine as amino acid moiety. Thus the prior art leads the skilled person to expect that L-aspartic acid di-esters are not useful in the design of novel ProTides. However, the present invention is based on the unexpected finding that the synthesis of higher esters (in particular C₅-esters) of L-aspartic acid, L-glutamic acid and L-serine show unexpected biological properties, in particular have significant antiviral activity.

### SUMMARY OF THE INVENTION

The present invention relates to novel phosphoramidates of nucleosides, and their use as agents for treating viral diseases. It is based on the unexpected finding that certain combinations of substituents in the phosphoramidate part of the nucleoside prodrug, said combinations not being suggested by the prior art, show unexpected biological properties, in particular have significant antiviral activity. The present invention furthermore relates to the use of the compounds of this invention for treating proliferative diseases such as cancer.

Preferred statements (features) and embodiments of the compounds, methods and uses of this invention are set herein below. Each statement and embodiment of the invention so defined may be combined with any other statement and/or embodiments unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below numbered aspects and embodiments 1 to 47 with any other statement and/or embodiments.
1. A compound of formula I:

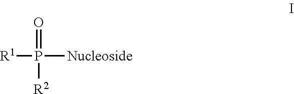

wherein
   Nucleoside can be any natural nucleoside or a nucleoside analogue;
   R¹ has the general formula II:

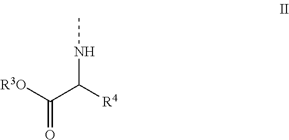

wherein
   R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, X—NH—R⁶, X—S—R⁶, wherein X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio;
R⁶ is selected from the group consisting of formyl, acyl, thioacyl, amide, thioamide, sulfonyl, sulfinyl, carboxylate, thiocarboxylate, amino-substituted acyl, alkoxyalkyl, C₃-C₁₀ cycloalkyl-alkyl, C₃₋₁₀ cycloalkyl, (di)alkylaminoalkyl, arylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁-C₇ alkyl, C₂-C₇ alkenyl, C₂-C₇ alkynyl, arylalkenyl, aryloxyalkyl, arylalkyl, aryl, arylaminoalkyl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino;
   R² is Y—Ar
wherein Y is O, NH or S; and Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
or R² has the general formula II:

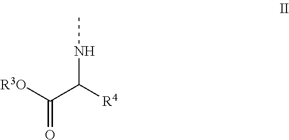

wherein
   R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, X—NH—R⁶, X—S—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁-C₇ alkyl, thio C₃-C₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine alkylhydrazino, phenylhydrazino, sulfonyl, sulfinyl and sulfonamido;
R⁵ is selected from the group consisting of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio;
R⁶ is selected from the group consisting of formyl, acyl, thioacyl, amide, thioamide, sulfonyl, sulfinyl, carboxylate, thiocarboxylate, amino-substituted acyl, alkoxyalkyl, C₃-C₁₀ cycloalkyl-alkyl, C₃₋₁₀ cycloalkyl, (di)alkylaminoalkyl, arylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, ω-carboxylic ester-alkyl, halo C₁-C₇ alkyl, C₂-C₇ alkenyl, C₂-C₇ alkynyl, arylalkenyl, aryloxyalkyl, arylalkyl, aryl, arylaminoalkyl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino; and
wherein R¹ and R² can be identical or different; and
when R³ is C₁-C₁₀ alkyl and R⁵ comprises an alkoxy moiety, R⁵ comprises at least 3 carbon atoms;
and/or a pharmaceutical acceptable addition salt thereof and/or a stereoisomer thereof and/or a solvate thereof and/or prodrugs thereof,
provided that said compound is not (2S,2′S)-1,4-Dibenzyl 2,2′-((((2R,3R,4R,5R)-5-(2-amino-6-methoxy-9H-purin-9-yl)-3,4-dihydroxy-4-methyltetrahydrofuran-2-yl)methoxy)phosphoryl)bis(azanediyl)disuccinate.
2. The compound according to statement 1, wherein the nucleoside is selected from the group consisting of 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, Emtricitabine, AZT, BVDU, HPMC, PMEA, PMPA, 4′-α-azido-cytidine, 2′deoxy-2′-α-guanosine, 5-F-uridine, gemcitabine, cytarabine, fludarabine, cladribine, Vidaza, clofarabine, nelarabine, decitabine, troxacitabine, and thiarabine.
3. The compound according to statements 1 or 2, wherein the nucleoside is 2′-β-C-Me-Cytidine, 2′-β-C-Me-Uridine, or 2′-deoxy-2′-α-fluoro-2′-β-C-methyluridine.
4. The compound according to any one of statements 1 to 3, wherein R² is O—Ar.
5. The compound according to any one of statements 1 to 3, wherein R² is O-Phenyl.
6. The compound according to any one of statements 1 to 5, wherein
   R¹ has the general formula II:

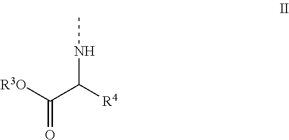

wherein R³ is C₁-C₁₀ alkyl.
7. The compound according to any one of statements 1 to 6, wherein R³ is C₃-C₁₀ alkyl.
8. The compound according to any one of statements 1 to 7, wherein R⁴ is X—COR⁵, wherein X is C₁-C₁₀ alkyl and wherein R⁵ is selected from the group consisting of C₁-C₇ alkoxy, C₃-C₁₀ cycloalkoxy, aryloxy, arylalkyloxy.
9. The compound according to any one of statements 1 to 8, wherein R⁴ is X—COR⁵, wherein X is C₁-alkyl or C₂-alkyl and wherein R⁵ is selected from the group consisting of C₃-C₇ alkoxy or aryl-(C₁-C₂)alkyloxy.
10. A compound selected from the group consisting of: 2′-C-methylcytidine-5′-[phenyl-bis(methoxy-aspartyl)]phosphate; 2′-C-methylcytidine-5′-[phenyl-(α-methoxy-β-benzyloxy-aspartyl)]phosphate; 2′-C-Methylcytidine-5′-[1-phenyl-bis(isopropyl-aspartyl)]phosphate; 2′-C-methyluridine-5′-[phenyl-bis(methoxy-aspartyl)]phosphate; 2′-C-methyluridine-5′-[phenyl-(α-methoxy-β-benzyloxy-aspartyl)]phosphate; 2′-C-Methyl-uridine-5′-[1-phenyl-bis(isopropyl-aspartyl)]phosphate; 2′-C-Methyl-uridine-5′-[phenyl-bis(n-butyl-aspartyl)]phosphate; 2′-C-Methyl-uridine-5′-[phenyl-bis(amyl-aspartyl)]phosphate; 2′-C-Methyl-uridine-5′-[phenyl-bis(isoamyl-aspartyl)]phosphate; 2′-C-Methylcytidine-5′-[phenyl-bis(n-butyl-aspartyl)]phosphate; 2′-C-Methyl-cytidine-5′-[phenyl-bis(amyl-aspartyl)]phosphate; 2′-C-Methylcytidine-5′-[phenyl-bis(isoamyl-aspartyl)]phosphate, and 2′-deoxy-2′-fluoro-2′-C-methyl-uridine-5′-[phenyl-bis(isoamyl-aspartyl)]phosphate.
11. A compound of formula I:

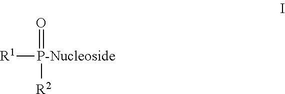

wherein
   Nucleoside is a natural nucleoside or a nucleoside analogue;
   R¹ has the general formula II:

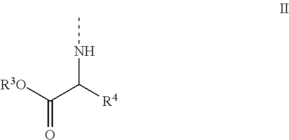

wherein
   R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, wherein
   X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of C₁-C₇ alkoxy, aryloxy, C₃-C₁₀ cycloalkoxy, arylalkyloxy;
R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, aryl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino;
   R² is Y—Ar
wherein Y is O; and Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
wherein when R³ is C₁-C₁₀ alkyl and R⁵ comprises an alkoxy moiety, R⁵ comprises at least 3 carbon atoms;
and/or a pharmaceutical acceptable addition salt thereof and/or a stereoisomer thereof and/or a solvate thereof and/or prodrugs thereof.
12. The compound according to any one of statements 1 or 11, wherein
   Nucleoside is a natural nucleoside or a nucleoside analogue;
   R¹ has the general formula II:

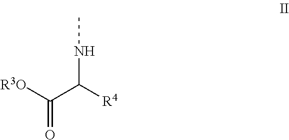

wherein
   R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of C₁-C₇ alkoxy, aryloxy, C₃-C₁₀ cycloalkoxy, arylalkyloxy;
R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, aryl;
   R² is Y—Ar
wherein Y is O; and Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
wherein when R³ is C₁-C₁₀ alkyl and R⁵ comprises an alkoxy moiety, R⁵ comprises at least 3 carbon atoms.
13. The compound according to any one of statements 1, 4 to 9, 11 to 12, wherein the nucleoside is selected from the group consisting of 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, 2′deoxy-2′-α-fluoro-guanosine, gemcitabine, 2′deoxy-2′-α-fluoro-uridine or 2′deoxy-2′-α-chloro-uridine.
14. The compound according to any one of statements 1, 4 to 9, 11 to 13, wherein the nucleoside is 2′-β-C-Me-Cytidine, 2′-β-C-Me-Uridine, 2′-deoxy-2′-α-fluoro-2′-β-C-methyluridine, gemcitabine, 2′deoxy-2′-α-fluoro-uridine or 2′deoxy-2′-α-chloro-uridine.
15. The compound according to any one of statements 1 to 9, 11 to 14, wherein the nucleoside is selected from the group consisting of 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, gemcitabine.
16. The compound according to any one of statements 1, 11, 12, having formula IA,

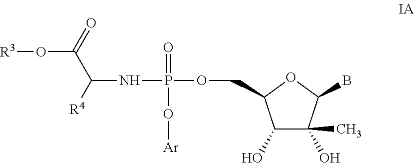

wherein
B is a purine or a pyrimidine base;
Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;
R⁴ is selected from the group consisting of X—COR⁵, X—O—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of C₁-C₇ alkoxy, aryloxy, C₃-C₁₀ cycloalkoxy, arylalkyloxy;
R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, aryl.
17. A compound having formula IB;

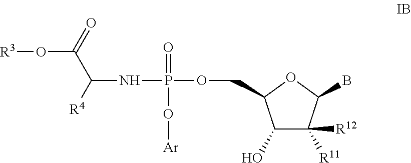

wherein
R¹¹ is OH or halogen, and
when R¹¹ is OH, R¹² is selected from the group consisting of C₁₋₁₀ alkyl, C₂₋₁₀ alkenyl, C₂₋₁₀ alkynyl;
when R¹¹ is a halogen, R¹² is selected from the group consisting of H, halogen, C₁₋₁₀ alkyl, C₂₋₁₀ alkenyl, C₂₋₁₀ alkynyl; in an embodiment at least one of R¹¹ or R¹² is halogen;
B is a purine or a pyrimidine base;
Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;
R¹⁴ is selected from the group consisting of X—COR¹⁵, X—O—R¹⁶, wherein
   X is aryl, heteroaryl, C₁-C₁₀ alkyl, C₁-C₁₀ alkyl, C₂-C₁₀ alkenyl, or C₃-C₈-cycloalkyl, and
wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R¹⁵ is R¹⁷—O—, wherein R¹⁷ is selected from the group consisting of aryl, heteroaryl, C₁-C₁₀alkyl, C₃-C₁₀-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; preferably R¹⁷ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; preferably R¹⁷ is C₁-C₇ alkyl, aryl, C₃-C₁₀ cycloalkyl, arylalkyl; preferably R¹⁷ is C₁-C₇ alkyl, aryl, C₃-C₁₀ cycloalkyl, arylalkyl; preferably R¹⁷ is C₁-C₇ alkyl, aryl, C₃-C₈ cycloalkyl, arylalkyl;
R¹⁶ is —CO—R¹⁸ or is selected from the group consisting of aryl, heteroaryl, C₁-C₁₀ alkyl, C₃-C₁₀-cycloalkyl, C₃-C₁₀cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; wherein R¹⁸ is selected from the group consisting of aryl, heteroaryl, C₁-C₁₀ alkyl, C₃-C₁₀-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; preferably R¹⁶ is —CO—R¹⁸ or is selected from the group consisting of aryl, heteroaryl, C₁-C₁₀ alkyl, C₃-C₁₀-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; wherein R¹⁸ is selected from the group consisting of aryl, heteroaryl, C₁-C₁₀ alkyl, C₃-C₁₀-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl; preferably R¹⁶ is alkoxyalkyl, C₃-C₁₀ cycloalkyl-alkyl, C₃₋₁₀ cycloalkyl, halo C₁-C₇ alkyl, C₂-C₇ alkenyl, C₂-C₇ alkynyl, arylalkyl, aryl; preferably R¹⁶ is alkoxyalkyl, C₃-C₈ cycloalkyl-alkyl, C₃₋₈ cycloalkyl, halo C₁-C₇ alkyl, C₂-C₇ alkenyl, C₂-C₇ alkynyl, arylalkyl, aryl;
and/or a pharmaceutical acceptable addition salt thereof and/or a stereoisomer thereof and/or a solvate thereof and/or prodrugs thereof.
18. The compound according to any one of statements 1, 11, 12, 17, having formula IB

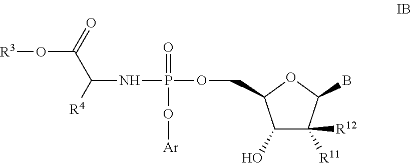

wherein
R¹¹ is OH or halogen, and
when R¹¹ is OH, R¹² is selected from the group consisting of C₁₋₁₀ alkyl, C₂₋₁₀ alkenyl, C₂₋₁₀ alkynyl;
when R¹¹ is a halogen, R¹² is selected from the group consisting of H, halogen, C₁₋₁₀ alkyl, C₂₋₁₀ alkenyl, C₂₋₁₀ alkynyl;
B is a purine or a pyrimidine base;
Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;
R¹⁴ is selected from the group consisting of X—COR¹⁵, X—O—R¹⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R¹⁵ is R¹⁷—O—, wherein R¹⁷ is selected from the group consisting of C₁-C₇ alkyl, aryl, C₃-C₁₀ cycloalkyl, arylalkyl;
R¹⁶ is selected from the group consisting of alkoxyalkyl, C₃-C₁₀ cycloalkyl-alkyl, C₃₋₁₀ cycloalkyl, halo C₁-C₇ alkyl, C₂-C₇ alkenyl, C₂-C₇ alkynyl, arylalkyl, aryl.
19. The compound according to any one of statements 1 to 9, 11 to 18, wherein Ar is Phenyl.
20. The compound according to any one of statements 1 to 6, 8, 9, 11 to 19, wherein R³ is C₁-C₁₀ alkyl.
21. The compound according to any one of statements 1 to 9, 11 to 20, wherein R³ is C₃-C₁₀ alkyl.
22. The compound according to any one of statements 11 to 16, wherein R⁴ is X—COR⁵, wherein X is C₁-C₁₀ alkyl and wherein R⁵ is selected from the group consisting of C₁-C₇ alkoxy, C₃-C₁₀ cycloalkoxy, aryloxy, arylalkyloxy.
23. The compound according to any one of statements 11 to 16, 22, wherein R⁴ is X—COR⁵ wherein X is C₁-alkyl or C₂-alkyl and wherein R⁵ is selected from the group consisting of C₃-C₇ alkoxy or aryl-(C₁-C₂)alkyloxy.
24. The compound according to any one of statements 1 to 9, 11 to 23, wherein X is CH₂.
25. The compound according to any one of statements 17 to 21, 24, wherein R¹⁴ is X—COOR¹⁷.
26. The compound according to statements 25, wherein R¹⁷ is C₅ alkyl.
27. The compound according to any one of statements 17 or 18, wherein R¹¹ is OH and R¹² is CH₃.
28. The compound according to any one of statements 17 or 18, wherein R¹¹ is F and R¹² is CH₃.
29. The compound according to any one of statements 17 or 18, wherein R¹¹ is F and R¹² is H.
30. The compound according to any one of statements 17 or 18, wherein R¹¹ is Cl and R¹² is H.
31. The compound according to any one of statements 17 or 18, wherein R¹¹ is Cl and R¹² is CH₃.
32. The compound according to any one of statements 17 or 18, wherein R¹¹ and R¹² are both F.
33. The compound according to any one of statements 17 or 18, wherein R¹¹ and R¹² are both Cl.
34. The compound according to any one of statements 16 to 33, wherein B is a pyrimidine base of structural formula III or a purine base of structural formula IV;

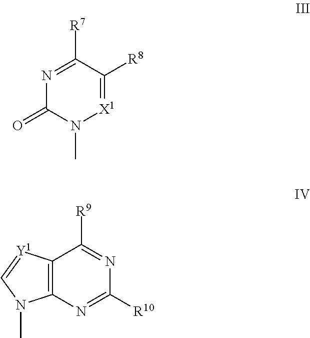

wherein:
R⁷ and R⁹ are independently selected from the group consisting of H, —OH, —SH, —NH₂, and —NH-Me;
R⁸ and R¹⁰ are independently selected from the group consisting of H, methyl, ethyl, isopropyl, hydroxyl, amino, ethylamino, trifluoromethyl, cyano and halogen; and
X¹ and Y¹ are independently selected from CH and N.
35. The compound according to any one of statements 16 to 33, wherein B is a pyrimidine or purine bases selected from the group comprising adenine, thymine, cytosine, uracyl, guanine and 2,6-diaminopurine and analogues thereof derived by replacement of a CH moiety by a nitrogen atom or vice versa or both; and derivative thereof wherein ring substituents are either incorporated, removed, or modified by substituents selected from the group comprising halogen, hydroxyl, amino, (C₁-C₆)alkyl and others.
36. A compound selected from the group consisting of: 2′-C-methylcytidine-5′-[phenyl-bis(methoxy-L-aspartyl)]phosphate; 2′-C-methylcytidine-5′-[phenyl-(α-methoxy-β-benzyloxy-L-aspartyl)]phosphate; 2′-C-Methylcytidine-5′-[1-phenyl-bis(isopropyl-L-aspartyl)]phosphate; 2′-C-methyluridine-5′-[phenyl-bis(methoxy-L-aspartyl)]phosphate; 2′-C-methyluridine-5′-[phenyl-(α-methoxy-β-benzyloxy-L-aspartyl)]phosphate; 2′-C-Methyl-uridine-5′-[1-phenyl-bis(isopropyl-L-aspartyl)]phosphate; 2′-C-Methyl-uridine-5′-[phenyl-bis(n-butyl-L-aspartyl)]phosphate; 2′-C-Methyl-uridine-5′-[phenyl-bis(amyl-aspartyl)]phosphate; 2′-C-Methyl-uridine-5′-[phenyl-bis(isoamyl-aspartyl)]phosphate; 2′-C-Methylcytidine-5′-[phenyl-bis(n-butyl-L-aspartyl)]phosphate; 2′-C-Methyl-cytidine-5′-[phenyl-bis(amyl-L-aspartyl)]phosphate; 2′-C-Methylcytidine-5′-[phenyl-bis(isoamyl-L-aspartyl)]phosphate; 2′-deoxy-2′-fluoro-2′-C-methyl-uridine-5′-[phenyl-bis(isoamyl-L-aspartyl)]phosphate; Gemcitabine-5′-[phenyl-bis(isoamyl-L-aspartyl)]phosphate; Gemcitabine-5′-[phenyl-(4-benzyl-1-isoamyl-L-aspartyl)]phosphate; Gemcitabine-5′-[phenyl-(1-benzyl-4-isoamyl-L-aspartyl)]phosphate; Gemcitabine-5′-[phenyl-bis(ethyl-L-glutamyl)]phosphate; Gemcitabine-5′-[phenyl-bis(isoamyl-L-glutamyl)]phosphate; 2′-deoxy-2′-α-fluoro-uridine-5′-[phenyl-bis(isoamyl-L-aspartyl)]phosphate; 2′-C-Methyl-uridine-5′-[phenylbis(methoxy-L-glutamyl)]phosphate; 2′-C-Methyl-uridine-5′-[phenylbis(isoamyl-L-glutamyl)]phosphate; 2′-C-Methyl-uridine-5′-[phenyl(α-methoxy-β-O-benzyl-L-serine)]phosphate; 2′-C-Methyl-uridine-5′-[phenyl(α-isoamyl-β-O-benzyl-L-serine)]phosphate; 2′-Deoxy-2′-chlorouridine-5′-[phenyl-bis(isoamyl-L-aspartyl)]phosphate.
37. A compound according to any one of statements 1 to 36 for use as a medicine.
38. A compound according to any one of statements 1 to 36 for use as a medicine for the prevention or treatment of a viral infection in an animal, mammal or human.
39. The compound according to statement 38, wherein said viral infection is an infection with HIV, HCV, HBV, RSV, dengue virus, influenza virus, West Nile encephalitis virus, Japanese encephalitis virus, yellow fever virus, poliovirus, CMV, adenovirus, parainfluenza, rhinovirus, BK virus, Powasen virus, Rift Valley fever virus, Tacaribe virus, Venezuelan equine encephalitis virus, SARS coronavirus, and/or HSV.
40. The compound according to statement 38 or 39, wherein said viral infection is an infection with HCV, Dengue virus, West-Nile virus, Yellow Fever virus, Japanese encephalitis virus, Powasen virus, Rift Valley fever virus, Tacaribe virus, Polio virus, Venezuelan equine encephalitis virus, RSV, Influenza, HIV, SARS coronavirus.
41. The compound according to statement 38, wherein said viral infection is an infection with HCV, HIV, RSV, dengue virus, influenza virus, West Nile encephalitis virus, Japanese encephalitis virus, yellow fever virus, and/or poliovirus.
42. A compound according to statement 38, wherein said viral infection is an infection of HIV, HCV, HBV, RSV, dengue virus, influenza virus, CMV, adenovirus, parainfluenza, rhinovirus, BK virus, and/or HSV.
43. A compound according to any one of statements 1 to 36 for use as a medicine for the prevention or treatment of a proliferative disorder such as cancer in an animal, mammal or human.
44. A pharmaceutical composition comprising a therapeutically effective amount of a compound according to any one of statements 1 to 36 and one or more pharmaceutically acceptable excipients.
45. The pharmaceutical composition according to statement 44, further comprising one or more biologically active drugs being selected from the group consisting of antiviral drugs and/or antiproliferative drugs.
46. A method of prevention or treatment of a viral infection in an animal, mammal or human, comprising the administration of a therapeutically effective amount of a compound according to any one of statements 1 to 36, optionally in combination with one or more pharmaceutically acceptable excipients.
47. A method of prevention or treatment of a proliferative disorder in an animal, mammal or human, comprising the administration of a therapeutically effective amount of a compound according to any one of statements 1 to 36, optionally in combination with one or more pharmaceutically acceptable excipients.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DETAILED DESCRIPTION OF THE INVENTION

According to one embodiment, the present invention encompasses compounds of the general formula

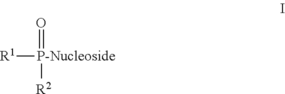

wherein
Nucleoside can be any natural nucleoside or a nucleoside analogue;
R¹ has the general formula II:

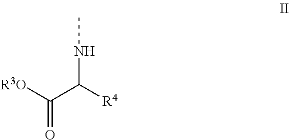

wherein
   R³ is selected from the group consisting of aryl, heteroaryl, C₁-C₁₀ alkyl, C3-C8-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, X—NH—R⁶, X—S—R⁶, wherein
      X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine alkylhydrazino, phenylhydrazino, sulfonyl, sulfinyl and sulfonamido;
      R⁵ is selected from the group of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio;
      R⁶ is selected from the group of formyl, acyl, thioacyl, amide, thioamide, sulfonyl, sulfinyl, carboxylate, thiocarboxylate, amino-substituted acyl, alkoxyalkyl, C₃-C₁₀ cycloalkyl-alkyl, C₃₋₁₀ cycloalkyl, (di)alkylaminoalkyl, arylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁-C₇ alkyl, C₂-C₇ alkenyl, C₂-C₇ alkynyl, arylalkenyl, aryloxyalkyl, arylalkyl, aryl, arylaminoalkyl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino;
R² is Y—Ar
   wherein Y is O, NH or S; and Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
or R² has the general formula II

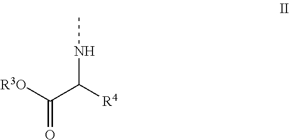

wherein
   R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   R⁴ is selected from the group consisting of X—COR⁵, X—O—R⁶, X—NH—R⁶, X—S—R⁶, wherein
      X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁-C₇ alkyl, thio C₃-C₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine alkylhydrazino, phenylhydrazino, sulfonyl, sulfinyl and sulfonamido;
      R⁵ is selected from the group consisting of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio;
      R⁶ is selected from the group of formyl, acyl, thioacyl, amide, thioamide, sulfonyl, sulfinyl, carboxylate, thiocarboxylate, amino-substituted acyl, alkoxyalkyl, C₃-C₁₀ cycloalkyl-alkyl, C₃₋₁₀ cycloalkyl, (di)alkylaminoalkyl, arylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, ω-carboxylic ester-alkyl, halo C₁-C₇ alkyl, C₂-C₇ alkenyl, C₂-C₇ alkynyl, arylalkenyl, aryloxyalkyl, arylalkyl, aryl, arylaminoalkyl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino; and wherein R¹ and R² can be identical or different; and

when R³ is C₁-C₁₀ alkyl and R⁵ comprises an alkoxy moiety, R³ or R⁵ comprises at least 3 carbon atoms;

and/or a pharmaceutical acceptable addition salt thereof and/or a stereoisomer thereof and/or a solvate thereof and/or a prodrug thereof,

provided that said compound is not (2S,2′S)-1,4-Dibenzyl 2,2′-((((2R,3R,4R,5R)-5-(2-amino-6-methoxy-9H-purin-9-yl)-3,4-di hydroxy-4-methyltetrahydrofuran-2-yl)methoxy)phosphoryl)bis(azanediyl)disuccinate.

Preferably, the invention encompasses a compound of formula I, wherein
Nucleoside is a natural nucleoside or a nucleoside analogue;
R¹ has the general formula II:

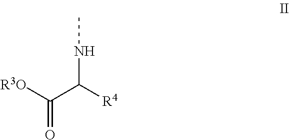

wherein
R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
R⁴ is selected from the group consisting of X—COR⁵, X—O—R⁶, wherein

X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;

R⁵ is selected from the group consisting of C₁-C₇ alkoxy, aryloxy, C₃-C₁₀ cycloalkoxy, arylalkyloxy;

R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, aryl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino;
R² is Y—Ar

wherein Y is O; and Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;

wherein when R³ is C₁-C₁₀ alkyl and R⁵ comprises an alkoxy moiety, R⁵ comprises at least 3 carbon atoms;

and/or a pharmaceutical acceptable addition salt thereof and/or a stereoisomer thereof and/or a solvate thereof and/or prodrugs thereof.

In formula I, the Nucleoside is preferentially a natural Nucleoside or a nucleoside analogue. In certain embodiments said Nucleoside consists of a sugar ring and a base (B), wherein said sugar ring includes a modified sugar moiety, known in the art, such as a hexitol nucleic acid (HNA), a cyclohexene nucleic acid (CeNA), a locked nucleic acid (LNA), an altritol nucleic acid (ANA) and a peptide nucleic acid (PNA).

Said base (B) is selected from the group of the pyrimidine and purine bases. Such bases include natural bases, such as adenine, thymine, cytosine, uracyl, guanine and modified bases or modifications of said natural bases. In certain embodiments of the present invention said base is a guanine, cytosine, adenine, thymine, cytosine, or uracyl. In a more specific embodiment of the present invention, said base is a cytosine or uracyl. In another specific embodiment of the present invention said base is an uracyl. In another specific embodiment of the present invention said base is a thymine. In another specific embodiment of the present invention said base is an adenine. In another specific embodiment of the present invention said base is a guanine.

In a particular embodiment, in formula I the Nucleoside is of the formula N:

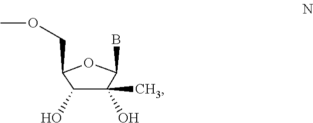

wherein B is a base which can be any base as described in the present invention and wherein the 5′O— is attached to the phosphorus atom P of formula I.

In another embodiment, in formula I the Nucleoside has the following structure:

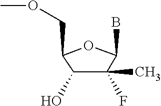

wherein B is a base which can be any base as described in the present invention and wherein the 5′O— is attached to the phosphorus atom P of formula I.

In another embodiment, in formula I the Nucleoside has the following structure

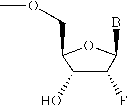

wherein B is a base which can be any base as described in the present invention and wherein the 5′O— is attached to the phosphorus atom P of formula I.

In another embodiment, in formula I the Nucleoside has the following structure

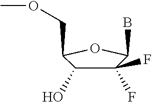

wherein B is a base which can be any base as described in the present invention and wherein the 5′O— is attached to the phosphorus atom P of formula I.

In another embodiment, in formula I the Nucleoside has the following structure

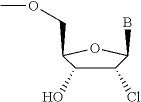

wherein B is a base which can be any base as described in the present invention and wherein the 5′O— is attached to the phosphorus atom P of formula I.

In another embodiment, in formula I the Nucleoside has the following structure

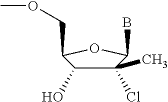

wherein B is a base which can be any base as described in the present invention and wherein the 5′O— is attached to the phosphorus atom P of formula I.

In another embodiment, in formula I the Nucleoside has the following structure

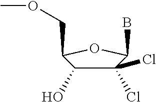

wherein B is a base which can be any base as described in the present invention and wherein the 5′O— is attached to the phosphorus atom P of formula I.

In formula I, the Nucleoside is preferentially selected from the group consisting of: 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, Emtricitabine, AZT, BVDU, HPMC, PMEA, PMPA, 4′-α-azido-cytidine, 2′deoxy-2′-α-guanosine, 5-F-uridine, gemcitabine, cytarabine, fludarabine, cladribine, Vidaza, clofarabine, nelarabine, decitabine, troxacitabine, and thiarabine. In some embodiments, the nucleoside, is preferably a 5-membered sugar ring attached to B, and is preferentially selected from the group consisting of: 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, 2′deoxy-2′-α-guanosine, gemcitabine, 2′-deoxy-2′-α-fluoro-uridine, 2′-deoxy-2′-chlorouridine. In a specific embodiment, said nucleoside is 2′-β-C-Me-Cytidine, 2′-β-C-Me-Uridine, 2′-deoxy-2′-α-fluoro-2′-β-C-methyluridine, 2′-deoxy-2′-α-fluoro-uridine, 2′,2′-difluorodeoxycytidine, 2′-deoxy-2′-chlorouridine. In a specific embodiment, said Nucleoside is 2′-β-C-Me-Cytidine or 2′-β-C-Me-Uridine. In a more specific embodiment said Nucleoside is 2′-β-C-Me-Cytidine. In another specific embodiment, said Nucleoside is 2′-β-C-Me-Uridine. In another specific embodiment, said Nucleoside is 2′,2′-difluorodeoxycytidine. In another specific embodiment, said nucleoside is 2′-deoxy-2′-chlorouridine.

In an embodiment, the present invention concerns a compound according to the invention, including the compound of formula I, wherein R² has the general formula II

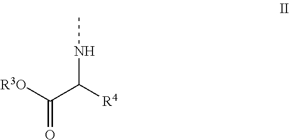

wherein R³ and R⁴ can have any of the values as described herein. In a more specific embodiment, said R³ is a C₁-C₁₀ alkyl. In another specific embodiment, said R³ is a C₃-C₁₀ alkyl.

In another specific embodiment, said R³ is an aryl. In another specific embodiment, said R⁴ is selected from the group consisting of X—COR⁵, X—O—R⁶, X—NH—R⁶, X—S—R⁶, wherein X, R⁵ and R⁶ can have any values as described herein. In a more specific embodiment, said R⁴ is X—COR⁵, wherein X is aryl, heteroaryl, C₁-C₁₀ alkyl, or C₃-C₈-cycloalkyl, more specifically said X is a C₁-C₆ alkyl, even more specifically said X is a C₁-C₃ alkyl or C₁-C₂ alkyl or —CH₂—, and wherein R⁵ is selected from the group consisting of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio. In a more specific embodiment R⁵ is C₁-C₇ alkoxy or C₃-C₁₀ cycloalkoxy; in a more specific embodiment R⁵ is C₃-C₇ alkoxy, in an even more specific embodiment R⁵ is C₃-C₅ alkoxy. In another specific embodiment, R⁵ is benzyloxy or phenyl-methoxy.

In another embodiment, the present invention concerns a compound according to the invention, including the compound of formula I, wherein R² is Y—Ar, wherein Y is O, NH or S; and Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy. In a more specific embodiment R² is O—Ar, wherein Ar is an aryl moiety as described hereinabove; and in a more specific embodiment said Ar is phenyl. In a specific embodiment of the present invention, the compound of formula I can have any value for R¹ as described herein and can have any Nucleoside as described herein, wherein R² is O-phenyl.

In an embodiment, the present invention concerns a compound according to the invention, including the compound of formula I, wherein R¹ has the general formula II:

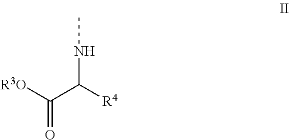

wherein
R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
R⁴ is selected from the group consisting of X—COR⁵, X—O—R⁶, X—NH—R⁶, X—S—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
   R₅ is selected from the group of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio;
   R⁶ is selected from the group of formyl, acyl, thioacyl, amide, thioamide, sulfonyl, sulfinyl, carboxylate, thiocarboxylate, amino-substituted acyl, alkoxyalkyl, C₃-C₁₀ cycloalkyl-alkyl, C₃₋₁₀ cycloalkyl, (di)alkylaminoalkyl, arylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, ω-carboxylic ester-alkyl, halo C₁-C₇ alkyl, C₂-C₇ alkenyl, C₂-C₇ alkynyl, arylalkenyl, aryloxyalkyl, arylalkyl, aryl, arylaminoalkyl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, arylalkylamino;
   when R³ is C₁-C₁₀ alkyl and R⁵ comprises an alkoxy moiety, R³ or R⁵ comprises at least 3 carbon atoms, preferably R⁵ comprises at least 3 carbon atoms;

and/or a pharmaceutical acceptable addition salt thereof and/or a stereoisomer thereof and/or a solvate thereof and/or prodrugs thereof;

provided that said compound is not (2S,2′S)-1,4-Dibenzyl 2,2′-((((2R,3R,4R,5R)-5-(2-amino-6-methoxy-9H-purin-9-yl)-3,4-dihydroxy-4-methyltetrahydrofuran-2-yl)methoxy)phosphoryl)bis(azanediyl)disuccinate.

In an embodiment, the compound has formula IA,

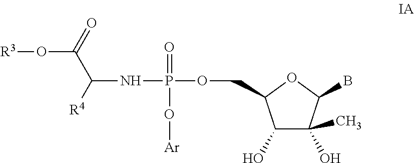

wherein

B is a purine or a pyrimidine base;

Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;

R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;

R⁴ is selected from the group consisting of X—COR⁵, X—O—R⁶, wherein

X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;

R⁵ is selected from the group consisting of C₁-C₇ alkoxy, aryloxy, C₃-C₁₀ cycloalkoxy, arylalkyloxy;

R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, aryl.

In another embodiment, the compound has formula IB,

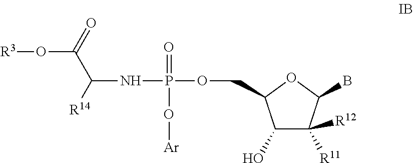

wherein

R¹¹ is OH or halogen, and

when R¹¹ is OH, R¹² is selected from the group consisting of C₁₋₁₀ alkyl, C₂₋₁₀ alkenyl, C₂₋₁₀ alkynyl;

when R¹¹ is a halogen, R¹² is selected from the group consisting of H, halogen, C₁₋₁₀alkyl, C₂₋₁₀ alkenyl, C₂₋₁₀ alkynyl;

B is a purine or a pyrimidine base;

Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;

R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;

R¹⁴ is selected from the group consisting of X—COR¹⁵, X—O—R¹⁶, wherein

X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;

R¹⁵ is R¹⁷—O—, wherein R¹⁷ is selected from the group consisting of C₁-C₇ alkyl, aryl, C₃-C₁₀ cycloalkyl, arylalkyl;

R¹⁶ is selected from the group consisting of alkoxyalkyl, C₃-C₁₀ cycloalkyl-alkyl, C₃₋₁₀ cycloalkyl, halo C₁-C₇ alkyl, C₂-C₇ alkenyl, C₂-C₇ alkynyl, arylalkyl, aryl.

In a more specific embodiment said R³ is C₁-C₁₀ alkyl. In another specific embodiment said R³ is C₃-C₁₀ alkyl. In another specific embodiment said R³ is C₁-C₅ alkyl. In yet another specific embodiment said R³ is C₃-C₅ alkyl.

In another specific embodiment, said R⁴ is selected from the group consisting of X—COR⁵, X—O—R⁶, X—NH—R⁶, X—S—R⁶, wherein X, R⁵ and R⁶ can have any values as described herein. In a more specific embodiment, said R⁴ is X—COR⁵, wherein X is aryl, heteroaryl, C₁-C₁₀ alkyl, or C₃-C₈-cycloalkyl, more specifically said X is a C₁-C₆ alkyl, even more specifically said X is a C₁-C₃ alkyl or C₁-C₂ alkyl or —CH₂—, and wherein R⁵ is selected from the group consisting of amino, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazine, alkylhydrazino, arylhydrazino, hydroxyl, C₁-C₇ alkoxy, C₃-C₁₀ cycloalkoxy, aryloxy, arylalkyloxy, oxyheterocyclic, heterocyclic-substituted alkyloxy, thio C₁-C₇ alkyl, thio C₃-C₁₀ cycloalkyl, thioaryl, thio-heterocyclic, arylalkylthio, heterocyclic-substituted alkylthio. In a more specific embodiment R⁵ is C₁-C₇ alkoxy or C₃-C₁₀ cycloalkoxy; in a more specific embodiment R⁵ is C₁-C₅ alkoxy, and in another more specific embodiment R⁵ is C₃-C₇ alkoxy, in an even more specific embodiment R⁵ is C₃-C₅ alkoxy. In another specific embodiment, R⁵ is aryl-(C₁-C₂)alkyloxy; in another more specific embodiment, R⁵ is benzyloxy or phenyl-methoxy.

In another embodiment, the present invention concerns a compound according to the invention, including the compound of formula I, or any subgroup thereof wherein Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy. In a more specific embodiment said Ar is phenyl. In a specific embodiment of the present invention, the compound of formula I and any subgroup thereof can have any value for R³ and R⁴ as described herein and can have any nucleoside as described herein, wherein Ar is phenyl.

In another specific embodiment, said R¹⁴ is selected from the group consisting of —X—COOR¹⁷, X—OCOR¹⁸, X—O—R¹⁶. In a more specific embodiment, said R¹⁴ is —X—COOR¹⁷. In a more specific embodiment, said R¹⁸ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl. In a more specific embodiment, said R¹⁶ is selected from the group consisting of alkoxyalkyl, C₃-C₁₀ cycloalkyl-alkyl, C₃₋₁₀ cycloalkyl, halo C₁-C₇ alkyl, C₂-C₇ alkenyl, C₂-C₇ alkynyl, arylalkyl, aryl. In a more specific embodiment, said R¹⁷ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl. In a more specific embodiment R¹⁷ is C₁-C₇ alkyl or C₃-C₁₀ cycloalkyl; in a more specific embodiment R¹⁷ is C₁-C₅ alkyl, and in another more specific embodiment R¹⁷ is C₃-C₇ alkyl, in an even more specific embodiment R¹⁷ is C₃-C₅ alkyl. In a yet more specific embodiment R¹⁷ is C₅ alkyl. In another specific embodiment, R¹⁷ is aryl-(C₁-C₂)alkyl; in another more specific embodiment, R¹⁷ is benzyl or phenyl-methyl.

In another specific embodiment, X is selected from the group consisting of aryl, heteroaryl, C₁-C₁₀ alkyl, C₂-C₁₀ alkenyl, C₂-C₁₀ alkynyl or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamide.

In a more specific embodiment, X is selected from the group consisting of aryl, heteroaryl, C₁-C₁₀ alkyl, or C₃-C₈-cycloalkyl, more specifically said X is a C₁-C₆ alkyl, even more specifically said X is a C₁-C₃ alkyl or C₁-C₂ alkyl or —CH₂—.

Special novel compounds in accordance with the present invention include each of the compounds whose preparation is described in the accompanying Examples, and pharmaceutically acceptable salts and solvates thereof. Examples of such novel compounds include intermediate molecules as described in the present invention such as di-isoamyl ester of L-aspartic acid (Example 13).

The present invention also concerns a compound having formula I, any subgroup thereof, or stereoisomeric forms thereof, for use as a medicine.

The present invention also concerns a compound having formula I any subgroup thereof, or stereoisomeric forms thereof, for use as a medicine for the prevention or treatment of viral disorders and oncological disorders in an animal, preferably in a mammal. In an embodiment, said disorder is a viral disorder, including a disease caused by a viral infection, for example an infection with HIV, HCV, HBV, RSV, dengue virus, influenza virus, CMV, adenovirus, parainfluenza, rhinovirus, BK virus, HSV, West-Nile virus, Yellow Fever virus, Japanese encephalitis virus, Powassen virus, Rift Valley fever virus, Tacaribe virus, Polio virus, Venezuelan equine encephalitis virus, SARS coronavirus, Norovirus, Ebolavirus; in another embodiment said disorder is an oncological disorder, which may be acute or chronic, including a proliferative disorder, especially cancer. In an embodiment, said mammal is a human being.

The present invention also concerns the use of the compounds of formula I, any subgroup thereof, or stereoisomeric forms thereof, for the manufacture of a medicament for the prevention or treatment of a viral disorder and/or an oncological disorder in an animal. In an embodiment, said animal is a mammal, preferably said mammal is a human being.

The present invention also concerns a pharmaceutical composition comprising a therapeutically effective amount of a compound having formula I, any subgroup thereof, or stereoisomeric forms thereof and one or more pharmaceutically acceptable excipients. Said composition may further comprise one or more biologically active drugs being selected from the group consisting of antiviral drugs, and antineoplastic drugs.

The present invention also concerns a method of prevention or treatment of a viral disorder in an animal, comprising the administration of a therapeutically effective amount of a compound having formula I, any subgroup thereof, or stereoisomeric forms thereof, optionally in combination with one or more pharmaceutically acceptable excipients.

The present invention also concerns a method of prevention or treatment of an oncological disorder in an animal, comprising the administration of a therapeutically effective amount of a compound having formula I, any subgroup thereof, or stereoisomeric forms thereof, optionally in combination with one or more pharmaceutically acceptable excipients.

For use in medicine, the salts of the compounds of formula (I) will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulphuric acid, methanesulphonic acid, fumaric acid, maleic acid, succinic acid, acetic acid, benzoic acid, citric acid, tartaric acid or phosphoric acid. Furthermore, where the compounds of the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope solvates of the compounds of formula (I) above.

Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

The compounds in accordance with the present invention are beneficial in the treatment and/or prevention of various animal, mammal or human ailments or diseases. These include viral diseases, such as diseases caused by a viral infection, for example an infection with HIV, HCV, HBV, RSV, dengue virus, influenza virus, CMV, adenovirus, parainfluenza, rhinovirus, BK virus, and/or HSV; and oncological disorders such as proliferative disorders (e.g. cancer).

Viral diseases include infections caused by various families of virus, including the Retroviridae, Flaviviridae, Picornaviridae. Various genera within the Retroviridae family include Alpharetrovirus, Betaretrovirus, Gammaretrovirus, Deltaretrovirus, Epsilonretrovirus, Lentivirus and Spumavirus. Members of the Lentivirus genus include human immunodeficiency virus 1 (HIV-1) and human immunodeficiency virus 2 (HIV-2). Various genera within the Flaviviridae family include Flavivirus, Pestivirus, Hepacivirus and Hepatitis G Virus. Members of the Flavivirus genus include Dengue fever virus, yellow fever virus, West Nile encephalitis virus and Japanese encephalitis virus. Members of the Pestivirus genus include bovine viral diarrhoea virus (BVDV), classical swine fever virus and border disease virus 2 (BDV-2). Members of the Hepacivirus genus include hepatitis C virus (HCV). Members of the Hepatitis G Virus genus include hepatitis G virus. Various genera within the Picornaviridae family include Aphthovirus, Avihepatovirus, Cardiovirus, Enterovirus, Erbovirus, Hepatovirus, Kobuvirus, Parechovirus, Sapelovirus, Senecavirus, Teschovirus and Tremovirus. Members of the Enterovirus genus include poliovirus, coxsackie A virus, coxsackie B virus and rhinovirus.

Oncological disorders, which may be acute or chronic, include proliferative disorders, especially cancer, in animals, including mammals, especially humans. Particular categories of cancer include haematological malignancy (including leukaemia and lymphoma) and non-haematological malignancy (including solid tumour cancer, sarcoma, meningioma, glioblastoma multiforme, neuroblastoma, melanoma, gastric carcinoma and renal cell carcinoma). Chronic leukaemia may be myeloid or lymphoid. Varieties of leukaemia include lymphoblastic T cell leukaemia, chronic myelogenous leukaemia (CML), chronic lymphocytic/lymphoid leukaemia (CLL), hairy-cell leukaemia, acute lymphoblastic leukaemia (ALL), acute myelogenous leukaemia (AML), myelodysplastic syndrome, chronic neutrophilic leukaemia, acute lymphoblastic T cell leukaemia, plasmacytoma, immunoblastic large cell leukaemia, mantle cell leukaemia, multiple myeloma, acute megakaryoblastic leukaemia, acute megakaryocytic leukaemia, promyelocytic leukaemia and erythroleukaemia. Varieties of lymphoma include malignant lymphoma, Hodgkin's lymphoma, non-Hodgkin's lymphoma, lymphoblastic T cell lymphoma, Burkitt's lymphoma, follicular lymphoma, MALT1 lymphoma and marginal zone lymphoma. Varieties of non-haematological malignancy include cancer of the prostate, lung, breast, rectum, colon, lymph node, bladder, kidney, pancreas, liver, ovary, uterus, cervix, brain, skin, bone, stomach and muscle.

The present invention also provides a pharmaceutical composition which comprises a compound in accordance with the invention as described above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

The quantity of a compound of use in the invention required for the prophylaxis or treatment of a particular condition or disease will vary depending on the compound chosen and the condition of the animal, mammal or human patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

### DEFINITIONS

The term “nucleoside analogue” as used herein refers to known nucleoside modifications wherein the sugar ring is modified or removed and therefore also comprises acyclic nucleosides. Nucleoside analogue examples wherein the natural sugar moiety is modified include but are not limited to hexitol nucleic acid (HNA), cyclohexene nucleic acids (CeNA), locked nucleic acids (LNA), altritol nucleic acids (ANA), peptide nucleic acids (PNA) and threose nucleic acids (TNA). Furthermore, halogenated (e.g. fluorinated or chlorinated) sugars, alkyl, alkenyl and alkynyl substituted sugars can be part of a nucleoside analogue.

The nucleoside or nucleoside analogue further comprises a base moiety (B) selected from the group of the pyrimidine and purine bases.

The term “pyrimidine and purine bases” as used herein includes, but is not limited to, adenine, thymine, cytosine, uracyl, guanine and 2,6-diaminopurine and analogues thereof. A purine or pyrimidine base as used herein includes a purine or pyrimidine base found in naturally occurring nucleosides as mentioned above. An analogue thereof is a base which mimics such naturally occurring bases in such a way that their structures (the kinds of atoms and their arrangement) are similar to the naturally occurring bases but may either possess additional or lack certain of the functional properties of the naturally occurring bases. Such analogues include those derived by replacement of a CH moiety by a nitrogen atom (e.g. 5-azapyrimidines such as 5-azacytosine) or vice versa (e.g., 7-deazapurines, such as 7-deazaadenine or 7-deazaguanine) or both (e.g., 7-deaza, 8-azapurines). By derivatives of such bases or analogues are meant those bases wherein ring substituents are either incorporated, removed, or modified by conventional substituents known in the art, e.g. halogen, hydroxyl, amino, (C₁-C₆)alkyl and others. Such purine or pyrimidine bases, and analogues thereof, are well known to those skilled in the art, e.g. as shown at pages 20-38 of WO 03/093290.

In particular purine and pyrimidine analogues B for the purpose of the present invention may be selected from the group comprising pyrimidine bases represented by the structural formula (III):

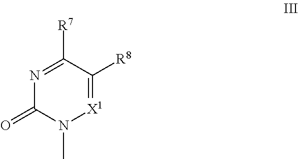

and purine bases represented by the structural formula (IV):

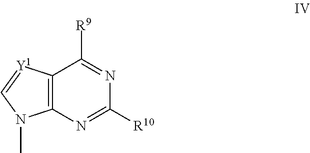

wherein:
R⁷ and R⁹ are independently selected from the group consisting of H, —OH, —SH, —NH₂, and —NH-Me;
R⁸ and R¹⁰ are independently selected from the group consisting of H, methyl, ethyl, isopropyl, hydroxyl, amino, ethylamino, trifluoromethyl, cyano and halogen; and
X¹ and Y¹ are independently selected from CH and N.

Just as a few non-limiting examples of pyrimidine analogues, can be named substituted uracils with the formula (III) wherein X¹ is CH, R⁷ is hydroxyl, and R⁸ is selected from the group consisting of methyl, ethyl, isopropyl, amino, ethylamino, trifluoromethyl, cyano, fluoro, chloro, bromo and iodo.

The term “alkyl” as used herein refers to a straight (normal) or branched (e.g. secondary, or tertiary) hydrocarbon chains having the number of carbon atoms as indicated (or where not indicated, preferably having 1-20, more preferably 1-6 carbon atoms). The term “C₁-C₆ alkyl” refers to such hydrocarbon chains having from 1 to 6 carbon atoms. Examples thereof are methyl, ethyl, 1-propyl, 2-propyl, 1-butyl, 2-methyl-1-propyl(i-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl.

As used herein and unless otherwise stated, the term “cycloalkyl” means a monocyclic saturated hydrocarbon monovalent radical having the number of carbon atoms as indicated (or where not indicated, preferably having 3-20, more preferably 3-10 carbon atoms, more preferably 3-8 or 3-6 carbon atoms). “C₃-C₈ cycloalkyl” refers to such monocyclic saturated hydrocarbon monovalent radical having from 3 to 8 carbon atoms, such as for instance cyclo-propyl, cyclobutyl, cyclopentyl, cyclohexyl, cycloheptyl, cyclooctyl.

The term “alkoxy” refers to the group alkyl-O—, where alkyl is as defined above. “(C₁-C₆) alkoxy” as used herein includes but is not limited to methoxy, ethoxy, propoxy, isopropoxy, butoxy, iso-butoxy, sec-butoxy, pentoxy, 3-pentoxy, or hexyloxy.

As used herein and unless otherwise stated, the term “halogen” or “halo” means any atom selected from the group consisting of fluorine (F), chlorine (Cl), bromine (Br) and iodine (I).

As used herein and unless otherwise stated, the term “Ar” or “aryl” means a monovalent unsaturated aromatic carbocyclic radical having one, two, three, four, five or six rings, preferably one, two or three rings, which may be fused or bicyclic. An aryl group may optionally be substituted by one, two, three or more substituents as set out in this invention with respect to optional substituents that may be present on the group Ar or aryl. Preferred aryl groups are: an aromatic monocyclic ring containing 6 carbon atoms; an aromatic bicyclic or fused ring system containing 7, 8, 9 or 10 carbon atoms; or an aromatic tricyclic ring system containing 10, 11, 12, 13 or 14 carbon atoms. Non-limiting examples of aryl include phenyl and naphthyl. Preferred substituent groups of Ar are independently selected from halogen, C₁-C₆ alkyl, C₁-C₆ alkoxy, hydroxy (—OH), acyl (R′—C(═O)—, acyloxy (R′—C(═O)—O—), nitro (—NO₂), amino (—NH₂), —SO₃H, —SH, —SR′, wherein R′ is an alkyl. Preferred Ar are phenyl, bromophenyl and naphthyl.

As used herein and unless otherwise stated, the term “heterocyclic” means a mono- or polycyclic, saturated or mono-unsaturated or polyunsaturated monovalent hydrocarbon radical having from 2 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, also including radicals wherein a heterocyclic ring is fused to one or more aromatic hydrocarbon rings for instance in the form of benzo-fused, dibenzo-fused and naphtho-fused heterocyclic radicals; within this definition are included heterocyclic radicals such as, but not limited to, diazepinyl, oxadiazinyl, triazolonyl, benzoquinolinyl, benzothiazinyl, benzothiazinonyl, benzoxa-thiinyl, benzodioxinyl, benzodithiinyl, benzoxazepinyl, benzothiazepinyl, benzodiazepine, benzodioxepinyl, benzodithiepinyl, benzoxazocinyl, benzo-thiazocinyl, benzodiazocinyl, benzoxathiocinyl, benzodioxocinyl, benzotrioxepinyl, benzoxathiazepinyl, benzoxadiazepinyl, benzothia-diazepinyl, benzotriazepinyl, benzoxathiepinyl, benzotriazinonyl, benzoxazolinonyl, azetidinonyl, hypoxanthinyl, azahypo-xanthinyl, bipyrazinyl, bipyridinyl, oxazolidinyl, benzodioxocinyl, benzopyrenyl, benzopyranonyl, benzophenazinyl, benzoquinolizinyl, dibenzo-carbazolyl, dibenzothiepinyl, dibenzoxepinyl, dibenzopyranonyl, dibenzothiazepinyl, dibenzisoquinolinyl, oxauracil, oxazinyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, naphthindazolyl, naphthindolyl, naphthothiazolyl, naphthothioxolyl, naphthoxindolyl, naphtho-triazolyl, naphthopyranyl, azabenzimidazolyl, azacycloheptyl, tetrahydrofuryl, tetrahydropyranyl, tetrahydro-pyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyl, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofutyl, furoyl, hydantoinyl, thienyl, indolyl, indazolyl, quinolyl, quinazolinyl, quinoxalinyl, carbazolyl, phenoxazinyl, phenothiazinyl, xanthenyl, purinyl, benzothienyl, naphtothienyl, pyranyl, pyronyl, benzopyronyl, isobenzofuranyl, chromenyl, phenoxathiinyl, indolizinyl, quinolizinyl, isoquinolyl, phthalazinyl, naphthiridinyl, cinnolinyl, pteridinyl, carbolinyl, phenanthrolinyl, phenazinyl, phenothiazinyl, imidazolinyl, imidazolidinyl, benzimidazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, homopiperazinyl, homopiperidinyl, thietyl, thietanyl, diazabicyclooctyl, diazetyl, diaziridinonyl, diaziridinethionyl, chromanyl, chromanonyl, thiochromanyl, thiochromanonyl, thiochromenyl, benzofuranyl, benzisothiazolyl, benzocarbazolyl, benzochromonyl, benzisoalloxazinyl, benzocoumarinyl, thiocoumarinyl, pheno-metoxazinyl, phenoparoxazinyl, phentriazinyl, thiodiazinyl, thiodiazolyl, indoxyl, thioindoxyl, benzodiazinyl (e.g. phthalazinyl), phtalidyl, phtalimidinyl, phtalazonyl, alloxazinyl, dibenzopyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof, wherein each carbon atom of said heterocyclic ring may furthermore be independently substituted with a substituent selected from the group consisting of halogen, nitro, C₁₋₇ alkyl (optionally containing one or more functions or radicals selected from the group consisting of carbonyl (oxo), alcohol (hydroxyl), ether (alkoxy), acetal, amino, imino, oximino, alkyloximino, amino-acid, cyano, carboxylic acid ester or amide, nitro, thio C₁₋₇ alkyl, thio C₃₋₁₀ cycloalkyl, C₁₋₇ alkylamino, cycloalkylamino, alkenylamino, cycloalkenylamino, alkynylamino, arylamino, arylalkyl-amino, hydroxylalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfonamido and halogen), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, amino, C₁₋₇ alkylamino, cycloalkylamino, alkenylamino, cycloalkenylamino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfhydryl, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, carboxylic acid or esters or thioesters or amides thereof, tricarboxylic acid or esters or thioesters or amides thereof; depending upon the number of unsaturations in the 3 to 10 atoms ring, heterocyclic radicals may be sub-divided into heteroaromatic (or “heteroaryl”) radicals and non-aromatic heterocyclic radicals; when a heteroatom of said non-aromatic heterocyclic radical is nitrogen, the latter may be substituted with a substituent selected from the group consisting of C₁₋₇ alkyl, C₃₋₁₀ cycloalkyl, aryl, arylalkyl and alkylaryl. Preferred heterocyclic rings are pyridyl, pyrimidyl, pyrazinyl pyridazinyl, furanyl, thienyl, quinolyl and isoquinolyl.

The following examples serve to merely illustrate the invention and should not be construed as limiting its scope in any way. While the invention has been shown in only some of its forms, it should be apparent to those skilled in the art that it is not so limited, but is susceptible to various changes and modifications without departing from the scope of the invention.

### EXAMPLES

### 1. Synthesis of the Nucleoside Building Blocks

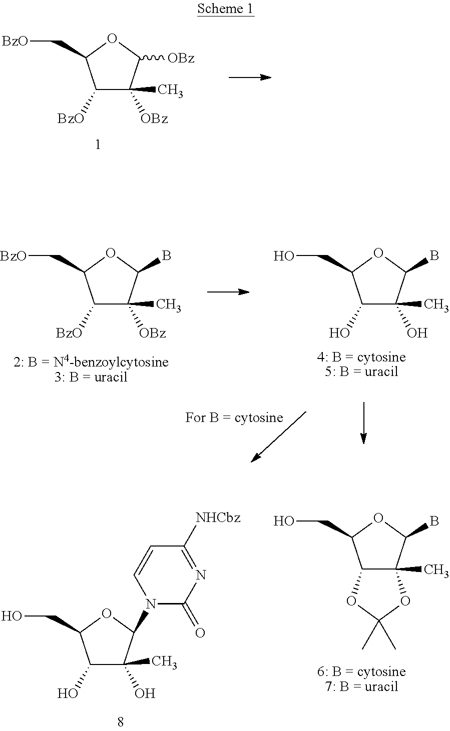

### Example 1

### Synthesis of 2′,3′,5′-Tri-O-benzoyl-N⁴-benzoyl-2′-C-methylcytidine (2)

This compound is prepared according to a literature procedure (J. Org. Chem. 1997, 62, 1754-1759).

TLC (EtOAc/Hexane, 1:1): R_{f}=0.47. Yield=80%.

HRMS (ESI+) calcd for C₃₈H₃₁N₃O₉Na [M+Na]⁺ 696.1953. found 696.1946.

### Example 2

### Synthesis of 2′-C-methylcytidine (4)

Saturated NH₃ in methanol (250 mL) was added to the compound of example 1 (5.4 g, 8.0 mmol) and was stirred overnight at room temperature. The reaction mixture was evaporated with silica gel and chromatographed on a silica gel column eluting with CH₂Cl₂/MeOH/NH₃ (8.3:1.5:0.2) to obtain the title compound as white solid (80%).

TLC (CH₂Cl₂/MeOH/NH₃, 8.3:1.5:0.2): R_{f}=0.13. Yield=80%.

¹H NMR (500 MHz, MeOD) δ: 8.13 (d, 1H, J_{6, 5}=7.5 Hz, H-6), 6.02 (s, 1H, H-1′), 5.89 (d, 1H, J_{5, 6}=7.5 Hz, H-5), 3.99-3.96 (dd, J=1.9 Hz, 12.45 Hz, 1H, H-5′), 3.93-3.91 (m, 1H, H-4′), 3.82-3.77 (m, 2H, H-3′ & H-5″), 1.10 (s, 3H, —CH₃).

¹³C NMR (125 MHz, MeOD) δ: 167.5 (C-4), 158.5 (C-2), 143.1 (C-6), 95.9 (C-5), 93.9 (C-1′), 83.8 (C-4′), 80.2 (C-2′), 73.7 (C-3′), 60.8 (C-5′), 20.5 (—CH₃).

HRMS (ESI+) calcd for C₁₀H₁₅N₃O₅Na [M+Na]⁺ 280.0904. found 280.0901.

### Example 3

### Synthesis of 2′-C-Methyl-2′,3′-O-(1-methylethylidene)-cytidine (6)

This compound is prepared according to a literature procedure (Bioorg. Med. Chem. Lett. 2009, 19, 1392-1395) and the authenticity of the molecule was judged by comparing the NMR data with the literature values.

TLC (CH₂Cl₂/MeOH, 9.0:1.0): R_{f}=0.48. Yield=90%.

¹H NMR (300 MHz, MeOD) δ: 7.95 (d, 1H, J_{6, 5}=7.53 Hz, H-6), 6.16 (s, 1H, H-1′), 5.92 (d, 1H, J_{5, 6}=7.53 Hz, H-5), 4.49 (d, J=2.97 Hz, 1H, H-3′), 4.26-4.23 (m, 1H, H-4′), 3.89-3.76 (ddd, 2H, H-5′ & H-5″), 1.57 (s, 3H, —CH₃), 1.40 (s, 3H, —CH₃), 1.24 (s, 3H, —CH₃).

¹³C NMR (75 MHz, MeOD) δ: 165.8, 156.4, 141.3, 113.2, 93.8, 93.6, 90.2, 85.9, 84.1, 61.1, 26.9, 25.9, 18.2

HRMS (ESI+) calcd for C₁₃H₂₀N₃O₅ [M+H]⁺ 298.1397. found 298.1402.

### Example 4

### Synthesis of 2′,3′,5′-Tri-O-benzoyl-2′-C-methyluridine (3)

This compound is prepared according to a literature procedure (J. Org. Chem. 1997, 62, 1754-1759) and the authenticity of the molecule was judged by comparing the NMR data with the literature values.

TLC (EtOAc/Hexane, 1:1): R_{f}=0.55. Yield=90%.

HRMS (ESI+) calcd for C₃₁H₂₆N₂O₉Na [M+Na]⁺ 593.1531. found 593.1533.

### Example 5

### Synthesis of 2′-C-methyluridine (5)

A similar synthetic and purification procedure as for the synthesis for example 2 was used.

TLC (CH₂Cl₂/MeOH/NH₃, 8.3:1.5:0.2): R_{f}=0.39. Yield=91%.

¹H NMR (600 MHz, MeOD) δ: 8.15 (d, 1H, J_{6, 5}=7.98 Hz, H-6), 5.95 (s, 1H, H-1′), 5.67 (d, 1H, J_{5, 6}=7.98 Hz, H-5), 3.99-3.96 (dd, J=2.1 Hz, 12.5 Hz, 1H, H-5′), 3.93-3.91 (m, 1H, H-4′), 3.84 (d, J=9.24 Hz, 1H, H-3′), 3.79-3.77 (dd, J=2.1 Hz, 12.5 Hz, 1H, H-5″), 1.15 (s, 3H, —CH₃).

¹³C NMR (150 MHz, MeOD) δ: 166.1 (C-4), 152.4 (C-2), 142.5 (C-6), 102.3 (C-5), 93.1 (C-1′), 83.8 (C-4′), 80.0 (C-2′), 73.3 (C-3′), 60.4 (C-5′), 20.1 (—CH₃).

HRMS (ESI+) calcd for C₁₀H₁₅N₂O₆ [M+H]⁺ 259.0925. found 259.0932.

### Example 6

### Synthesis of 2′-C-Methyl-2′,3′-O-(1-methylethylidene)-uridine (7)

This compound was prepared according to a literature procedure (Bioorg. Med. Chem. Lett. 2009, 19, 1392-1395) except for the quenching and purification method. After completion of the reaction by TLC, the reaction mixture was quenched by the addition of Et₃N and evaporated to dryness with silica gel and chromatographed on a silica gel column eluting with EtOAc/Hexane (50-90% EtOAc) to obtain the title compound as a white solid (81%).

TLC (CH₂Cl₂/MeOH, 9.0:1.0): R_{f}=0.54.

HRMS (ESI+) calcd for C₁₃H₁₉N₂O₆ [M+H]⁺ 299.1238. found 299.1239.

### Example 7

### Synthesis of 2′-C-methyl-N⁴-(benzyl-oxy-carbonyl)cytidine (8)

A suspension of compound 4 (60 mg, 0.23 mmol) in dry pyridine was prepared and cooled to 0° C. in an ice bath. Trimethyl silyl chloride (0.44 mL, 3.5 mmol) was added dropwise under an argon atmosphere. After 10 minutes, the ice bath was removed and the solution was left to stir at room temperature for 1.5 h. The reaction mixture was then cooled to 0° C. and benzyl chloroformate (0.13 mL, 1.2 mmol) was added slowly. After 10 minutes ice bath was removed and the solution was left to stir at room temperature for 2 h. Upon completion, the reaction was quenched by adding methanol (2 mL) at 0° C. and then left to stir at room temperature for overnight. To the solution was added saturated sodium bicarbonate (0.5 mL) and evaporated to dryness with repeated coevaporation using toluene. The residue was dissolved in methanol and evaporated with silica gel. The crude product was purified on silica gel column chromatography eluting with 0-4.5% methanol in dichloromethane to yield compound 8 as white solid (90%).

TLC (CH₂Cl₂/MeOH, 9.0:1.0): R_{f}=0.5.

¹H NMR (600 MHz, MeOD): δ=8.59 (d, 1H, J_{6, 5}=7.6 Hz, H-6), 7.42-7.29 (m, 6H, phenyl ring & H-5), 6.07 (s, 1H, H-1′), 5.22 (s, 2H, —CH₂Ph), 4.02-3.96 (m, 2H, H-5′ & H-4′), 3.86-3.80 (m, 2H, H-3′& H-5″), 1.10 (s, 3H, 2′-CH₃).

¹³C NMR (150 MHz, MeOD): δ=165.7 (C-4), 159.0 (C-2), 155.4 (CO—OCH₂Ph), 147.0 (C-6), 138.0 (phenyl C), 130.5, 130.3, 130.1 (phenyl C), 97.5 (C-5), 95.0 (C-1′), 84.8 (C-4′), 81.1 (C-2′), 74.0 (C-3′), 69.4 (—CH₂Ph), 61.2 (C-5′), 21.0 (2′-CH₃).

HRMS (ESI−) calcd for C₁₈H₂₂N₃O₇ [M−H]⁻ 390.1307. found 390.1305.

### 2. Synthesis of L-Aspartic Acid Di-Esters

### 2.1. Synthesis of Asymmetric Di-Esters of L-Aspartic Acid

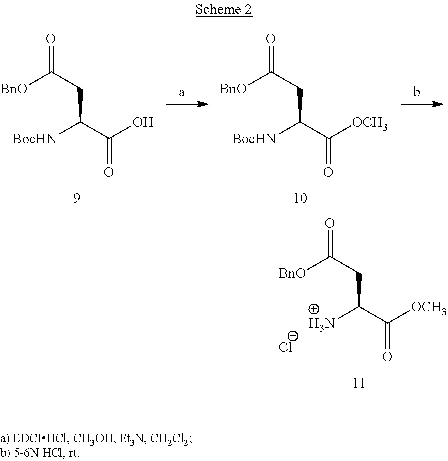 [SMILES: CC(C)(C)OC(=O)N[C@@H](CC(=O)OCc1ccccc1)C(=O)O ; COC(=O)[C@H](CC(=O)OCc1ccccc1)NC(=O)OC(C)(C)C ; [Cl-] ; COC(=O)[C@@H]([NH3+])CC(=O)OCc1ccccc1]

### Example 8

### Synthesis of Boc-L-Asp-(OBzl)-OMe (10)

Compound 9 (2 g, 6.2 mmol) was suspended in dry dichloromethane (50 mL) and allowed to cool to 0° C. in an ice bath. EDC.HCl (1.54 g, 8.0 mmol) was added and the reaction mixture was stirred for 30 min. Methanol (1 mL, 24.8 mmol) and Et₃N (2 mL) were then added to the mixture, and stirring was continued for 24 h at room temperature. Solvent was removed under reduced pressure. The residue was dissolved in ethyl acetate and washed with water and brine. The organic layer was dried over MgSO₄ and evaporated to dryness to obtain the crude product which was then purified by silica gel column chromatography eluting with EtOAc/Hexane (2:8) to obtain 10 (72%).

R_{f}=0.4 (EtOAc/hexane, 3:7).

¹H NMR (300 MHz, CDCl₃): δ=7.38-7.26 (m, 5H, Phenyl ring), 5.62 (d, 1H, —NH), 5.15-5.06 (m, 2H, CH₂ of Bn), 4.65-4.58 (m, 1H, Ha), 3.66 (s, 3H, CH₃), 3.04-2.83 (m, 2H, Hβ), 1.43 (s, 9H, t-Bu).

¹³C NMR (75 MHz, CDCl₃): δ=171.5, 170.7, 155.4, 135.6, 128.6, 128.5, 128.4, 128.3, 80.0, 66.6, 52.6, 50.1, 36.9, 28.3 ppm;

HRMS (ESI+) calcd for C₁₇H₂₃NO₆Na [M+Na]⁺ 360.1418. found 360.1418.

### Example 9

### Synthesis of LAsp-(OBz)-OMe (11) as hydrochloride salt

Compound 10 (1.5 g, 4.4 mmol) was dissolved in dichloromethane (15 mL). Approximately 5-6N HCl in isopropanol (1.8 mL) was added and the mixture was stirred at room temperature for 3-4 h. Upon completion, reaction mixture was evaporated to dryness and triturated with diethyl ether. The solid compound was then filtered and washed several times with diethyl ether to obtain compound 11 as a white solid (75%).

¹H NMR (300 MHz, CDCl₃): δ=8.82 (s, 3H, —NH₃), 7.31-7.27 (m, 5H, phenyl ring), 5.15 (s, 2H, CH₂), 4.66 (t, 1H, Ha), 3.65 (s, 3H, CH₃), 3.42-3.24 (m, 2H, Hβ).

¹³C NMR (75 MHz, CDCl₃): δ=170.2, 168.9, 135.6, 128.9, 128.7, 67.7, 53.8, 50.0, 34.5 ppm;

HRMS (ESI+) calcd for C₁₂H₁₆NO₄ [M+H]⁺ 238.1074. found 338.1072.

### 2.2. Synthesis of Symmetric Di-Esters of L-Aspartic Acid

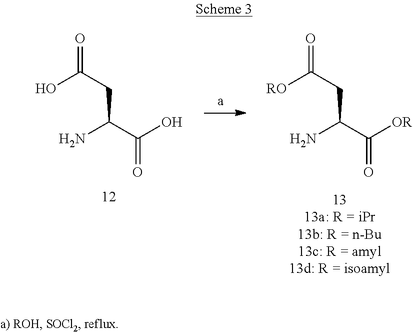

### Example 10

### Synthesis of the Di-Isopropyl Ester of L-Aspartic Acid (13a)

To a suspension of L-aspartic acid (2.6 g, 20.0 mmol) in anhydrous isopropanol (100 mL) thionyl chloride (10 mL, 139 mmol) was added dropwise at 0° C. under argon atmosphere. The mixture was allowed to come to RT and then refluxed for 8 h. After evaporation, solid residue was triturated with diethyl ether. The white solid product was then filtered and washed with diethyl ether to obtain the di-isopropyl ester of L-aspartic acid as hydrochloride salt (94%).

¹H NMR (300 MHz, DMSO-d₆): δ=8.67 (br s, 3H, —NH₃), 5.01-4.86 (m, 2H, —CH(CH₃)₂), 4.23 (t, 1H, H-α), 3.01-2.84 (dd, 2H, H-β′ & H-β″), 1.22-1.17 (a series of singlet, 12H, —CH₃) ppm.

¹³C NMR (75 MHz, DMSO-d₆): δ=168.7, 167.9, 70.1, 68.7, 48.6, 34.5, 21.6, 21.5, 21.4, 21.3 ppm.

HRMS (ESI+) calcd. for C₁₀H₂₀NO₄ [M+H]⁺ 218.1387. found 218.1387.

### Example 11

### Synthesis of the Di-n-Butyl Ester of L-Aspartic Acid (13b)

To a suspension of L-aspartic acid (1.6 g, 12.0 mmol) in anhydrous n-butanol (50 mL) thionyl chloride (6.2 mL, 85.2 mmol) was added dropwise at 0° C. under argon atmosphere. The mixture was allowed to come to room temperature and stirred for 12 h. The clear solution was then refluxed for 4 h. After evaporation, solid residue was triturated with diethyl ether. The off-white solid product was then filtered and washed several times with diethyl ether to obtain the di-n-butyl ester of L-aspartic acid (13b) as hydrochloride salt (94%).

¹H NMR (300 MHz, DMSO-d₆): δ=8.77 (br s, 3H, —NH₃⁺), 4.31 (t, 1H, H-α), 4.21-4.05 (m, 4H), 3.10-2.94 (2 dd, 2H, H-β′ & H-13″), 1.61-1.52 (m, 4H), 1.39-1.27 (m, 4H), 0.92-0.86 (m, 4H) ppm.

¹³C NMR (75 MHz, DMSO-d₆): δ=170.0, 169.1, 66.4, 65.4, 49.3, 35.0, 30.9, 30.8, 19.4, 19.3, 14.4, 14.3 ppm.

HRMS (ESI+) calcd. for C₁₂H₂₄NO₄ [M+H]⁺ 246.1699. found 246.1697.

### Example 12

### Synthesis of the Di-Amyl Ester of L-Aspartic Acid (13c)

To a suspension of aspartic acid (1.0 g, 7.5 mmol) in anhydrous amyl alcohol (25 mL) thionyl chloride (4.0 mL, 53.3 mmol) was added dropwise at 0° C. under argon atmosphere. The mixture was allowed to come to room temperature and stirred for 12 h. The suspension was then refluxed for 3 h. After evaporation, solid residue was triturated with diethyl ether. The off-white solid product was then filtered and washed several times with diethyl ether to obtain the di-amyl ester of L-aspartic acid (13c) as hydrochloride salt (82%).

¹H NMR (300 MHz, DMSO-d₆): δ=8.73 (br s, 3H, —NH₃′), 4.31 (t, 1H, H-α), 4.20-4.03 (m, 4H), 3.09-2.93 (2 dd, 2H, H-β′ & H-β″), 1.61-1.54 (m, 4H), 1.31-1.26 (m, 8H), 0.90-0.85 (m, 6H) ppm.

¹³C NMR (75 MHz, DMSO-d₆): δ=170.1, 169.2, 66.6, 65.6, 49.3, 35.0, 28.5, 28.4, 28.3, 28.2, 22.6, 22.5, 14.7 ppm.

HRMS (ESI+) calcd. for C₁₄H₂₈NO₄ [M+H]⁺ 274.2013. found 274.2007.

### Example 13

### Synthesis of the Di-Isoamyl Ester of L-Aspartic Acid (13d)

To a suspension of L-aspartic acid (1.0 g, 7.5 mmol) in anhydrous isoamyl alcohol (25 mL) thionyl chloride (4.0 mL, 53.3 mmol) was added dropwise at 0° C. under argon atmosphere. The mixture was allowed to come to room temperature and stirred for 12 h. The suspension was then just heated at 50° C. until a clear solution was obtained. After evaporation, the crude yellow liquid was triturated with hexane and kept at −78° C. for overnight. A jelly-type white precipitate was obtained and the hexane was immediately decanted carefully at that cold condition. Hexane was added again and kept at −78° C. until a jelly-type precipitate was formed and the above process was repeated several times to remove the impurities. The collective hexane was evaporated to one third and kept again at −78° C. and the aforementioned process is repeated to increase the final crop. Finally the white solid product was then washed several times with diethyl ether to obtain isoamyl ester of aspartic acid (8d) as hydrochloride salt (40%).

¹H NMR (300 MHz, DMSO-d₆): δ=8.60 (br s, 3H, —NH₃⁺), 4.33 (t, 1H, H-α), 4.24-4.07 (m, 4H), 3.06-2.89 (2 dd, 2H, H-β′ & H-β″), 1.69-1.58 (m, 2H), 1.51-1.45 (m, 4H), 0.90-0.86 (m, 12H) ppm.

¹³C NMR (75 MHz, DMSO-d₆): δ=170.1, 169.2, 65.2, 64.3, 49.3, 37.6, 37.4, 35.0, 25.3, 25.1, 23.2, 23.1, 23.0 ppm.

HRMS (ESI+) calcd. for C₁₄H₂₈NO₄ [M+H]⁺ 274.2013. found 274.2018.

### 3. Synthesis of 2′-C-Methyl Ribonucleoside Phosphoramidates

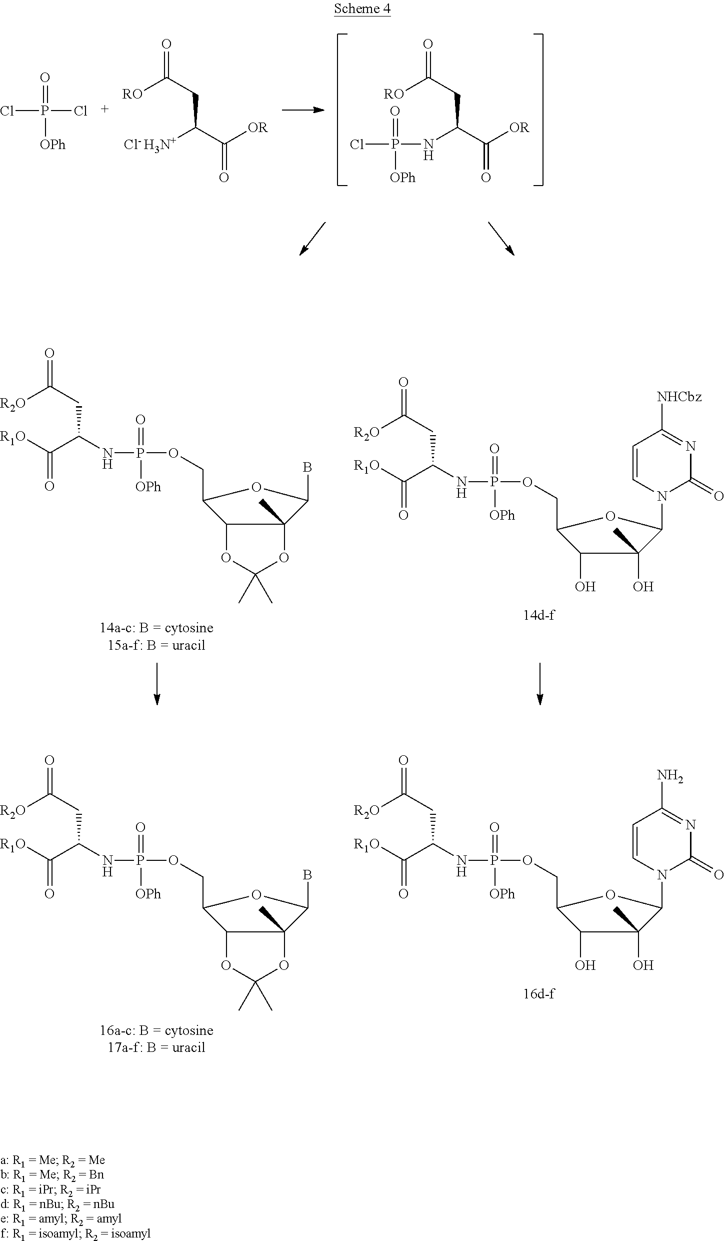

### Examples 14-25

### General Procedure for Synthesis of 14a-f and 15a-f

### Step 1

A solution/suspension of the appropriate L-aspartic acid di-ester hydrochloride (3.5 equiv) in anhydrous CH₂Cl₂ was prepared and cooled to −15° C. Dichlorophenyl phosphate (2.5 equiv) was added slowly. After 10 minutes, a solution of N-methylimidazole (10 equivalents) in dry CH₂Cl₂ was added dropwise. The mixture was allowed to reach room temperature slowly and left to stir for 10-12 h.

### Step 2

In a separate flask, a suspension of appropriately protected nucleoside (1 equiv of nucleoside 6, 7 or 8) in anhydrous CH₂Cl₂ was cooled to −5° C. With stirring, the solution prepared above was added slowly over a period of 1 h, keeping the temperature near −5° C. The cooling bath was removed, and the reaction was left to stir at room temperature (for nearly 4-6 h) until TLC indicates a reasonable amount of product formation. The reaction mixture was then evaporated to dryness under reduced pressure, and the residue was purified by column chromatography eluting with DCM/MeOH or EtOAc/Hexane in different proportion. Over all yield of the reaction is in the range of 30-90%.

The following compounds were made according to this procedure:

### Example 14

### 2′-C-methyl-2′,3′-O-isopropyliden-cytidine-5′-[phenyl-bis(methoxy-aspartyl)]phosphate (14a)

This compound was prepared starting from compound 6 and commercially available L-aspartic acid dimethyl ester hydrochloride.

Yield: 30%; R_{f}=0.25 (CH₂Cl₂/MeOH, 9.5:0.5);

³¹P NMR (121 MHz, CDCl₃): δ=2.98, 2.53 ppm;

HRMS (ESI+) calcd for C₂₅H₃₄N₄O₁₁P [M+H]⁺ 597.1956. found 597.1965.

### Example 15

### 2′-C-methyl-2′,3′-O-isopropyliden-cytidine-5′-[phenyl-(α-methoxy-β-benzyloxy-aspartyl)]phosphate (14b)

This intermediate was obtained starting from compounds 6 and 11.

Yield: 40%; R_{f}=0.27 (CH₂Cl₂/MeOH, 9.5:0.5); HRMS (ESI+) calcd for C₃₁H₃₈N₄O₁₁P [M+H]⁺ 673.2269. found 673.2270.

### Example 16

### 2′-C-Methyl-2′,3′-O-isopropyliden-cytidine-5′-[phenyl-bis(isopropyl-aspartyl)]phosphate (14c)

This intermediate was obtained starting from compounds 6 and 13a.

Yield=44%; R_{f}=0.42 (CH₂Cl₂/MeOH, 9.5:0.5)

³¹P NMR (121 MHz, CDCl₃) δ: 3.13 and 2.67.

HRMS (ESI+) calcd for C₂₉H₄₂N₄O₁₁P [M+H]⁺ 653.2582. found 653.2594.

### Example 17

### 2′-C-Methyl-N⁴-(benzyl-oxy-carbonyl)cytidine-5′-[phenyl-bis(n-butyl-aspartyl)]phosphate (14d)

This intermediate was obtained starting from compounds 10 and 13b.

Yield=50%; R_{f}=0.56 (CH₂Cl₂/MeOH, 9.5:0.5)

³¹P NMR (121 MHz, CDCl₃) δ: 3.30 and 2.89.

HRMS (ESI+) calcd for C₃₆H₄₈N₄O₁₃P [M+H]⁺ 775.2950. found 775.2947.

### Example 18

### 2′-C-Methyl-N⁴-(benzyl-oxy-carbonyl)cytidine-5′-[phenyl-bis(amyl-aspartyl)]phosphate (14e)

This intermediate was obtained starting from compounds 10 and 13c.

Yield=58%; R_{f}=0.55 (CH₂Cl₂/MeOH, 9.5:0.5)

³¹P NMR (121 MHz, CDCl₃) δ: 3.38 and 2.92.

HRMS (ESI−) calcd for C₃₈H₅₀N₄O₁₃P [M−H]⁻ 801.3117. found 801.3133.

### Example 19

### 2′-C-Methyl-N⁴-(benzyl-oxy-carbonyl)cytidine-5′-[phenyl-bis(isoamyl-aspartyl)]phosphate (14f)

This intermediate was obtained starting from compounds 10 and 13d.

Yield=58%; R_{f}=0.59 (CH₂Cl₂/MeOH, 9.5:0.5)

³¹P NMR (121 MHz, CDCl₃) δ: 3.32 and 2.93.

HRMS (ESI+) calcd for C₃₈H₅₂N₄O₁₃P [M+H]⁻ 803.3263. found 803.3268.

### Example 20

### 2′-C-methyl-2′,3′-O-isopropyliden-uridine-5′-[phenyl-bis(methoxy-aspartyl)]phosphate (15a)

This intermediate was obtained starting from compounds 7 and commercially available L-aspartic acid dimethyl ester hydrochloride.

Yield: 70%; R_{f}=0.47 (CH₂Cl₂/MeOH, 9.5:0.5);

³¹P NMR (121 MHz, CDCl₃): δ=3.17, 2.70 ppm;

HRMS (ESI−) calcd for C₂₅H₃₁N₃O₁₂P [M−H]⁻ 596.1651. found 596.1651.

### Example 21

### 2′-C-methyl-2′,3′-O-isopropyliden-uridine-5′-[phenyl-(α-methoxy-β-benzyloxy-aspartyl)]phosphate (15b)

This intermediate was obtained starting from compounds 7 and 11.

Yield: 76%; R_{f}=0.45 (CH₂Cl₂/MeOH, 9.5:0.5); HRMS (ESI−) calcd for C₃₁H₃₅N₃O₁₂P [M−H]⁻ 672.1964. found 672.1969.

### Example 22

### 2′-C-Methyl-2′,3′-O-isopropyliden-uridine-5′-[1-phenyl-bis(isopropyl-aspartyl)]phosphate (15c)

This intermediate was obtained starting from compounds 7 and 13a.

Yield=83%; R_{f}=0.75 (CH₂Cl₂/MeOH, 9.4:0.6)

¹H NMR (300 MHz, CDCl₃) δ: 9.49 (s, 1H, —NH), 7.62-7.47 (2 d, 1H, H-6), 7.37-7.14 (a series of multiplets, 5H, OPh), 6.12, 6.08 (2 s, 1H, H-1′), 5.72-5.58 (2 d, 1H, H-5), 5.09-4.92 (m, 2H, CH-iPr), 4.53-4.19 (m, 5H, H-5′, H-5″, —CH-Asp, H-4′, H-3′), 2.94-2.50 (m, 2H, —CH₂-Asp), 1.59 (s, 3H, —CH₃), 1.39 (s, 3H, —CH₃), 1.25-1.19 (m, 15H, —CH₃-iPr and —CH₃-2′). ³¹P NMR (121 MHz, CDCl₃) δ: 3.34 and 2.86.

HRMS (ESI−) calcd for C₂₉H₃₉N₃O₁₂P [M−H]⁻ 652.2277. found 652.2269.

### Example 23

### 2′-C-Methyl-2′,3′-O-isopropyliden-uridine-5′-[phenyl-bis(n-butyl-aspartyl)]phosphate (15d)

This intermediate was obtained starting from compounds 7 and 13b.

Yield=86%; R_{f}=0.45 (CH₂Cl₂/MeOH, 9.5:0.5)

³¹P NMR (121 MHz, CDCl₃) δ: 3.24 and 2.77.

HRMS (ESI−) calcd for C₃₁H₄₃N₃O₁₂P [M−H]⁻ 680.2590. found 680.2593.

### Example 24

### 2′-C-Methyl-2′,3′-O-isopropyliden-uridine-5′-[phenyl-bis(amyl-aspartyl)]phosphate (15e)

This intermediate was obtained starting from compounds 7 and 13c.

Yield=72%; R_{f}=0.35 (EtOAc/Hexane, 9.0:1.0)

³¹P NMR (121 MHz, CDCl₃) δ: 3.25 and 2.77.

HRMS (ESI+) calcd for C₃₃H₄₇N₃O₁₂P [M+H]⁺ 710.3048. found 710.3059.

### Example 25

### 2′-C-Methyl-2′,3′-O-isopropyliden-uridine-5′-[phenyl-bis(isoamyl-aspartyl)]phosphate (15f)

Yield=87%; R_{f}=0.65 (CH₂Cl₂/MeOH, 9.5:0.5)

³¹P NMR (121 MHz, CDCl₃) δ: 3.24 and 2.78.

HRMS (ESI+) calcd for C₃₃H₄₉N₃O₁₂P [M+H]⁺ 710.3048. found 710.3050.

### Examples 26-34

### General Procedure for Acetonide Deprotection

The protected phosphoramidate was dissolved in a solution of TFA/H₂O (8:2, 0.098 M) and was stirred at room temperature until TLC shows no starting material (typically 3-6 h). The reaction mixture was evaporated to dryness and coevaporated with toluene thrice. The solid material was then dissolved in methanol and evaporated with silica gel and purified by flash column chromatography eluting with CH₂Cl₂/MeOH in different proportion (generally 2-5% methanol in CH₂Cl₂) to obtain the required compound as white solid. Over all yield of the reaction is in the range of 42-86%.

The following compounds were made according to this procedure

### Example 26

### 2′-C-methylcytidine-5′-[phenyl-bis(methoxy-aspartyl)]phosphate (16a)

This compound was prepared from 14a.

Yield: 70%; R_{f}=0.15 (CH₂Cl₂/MeOH, 9.5:0.5);

¹H NMR (500 MHz, MeOD): δ=7.72-7.69 (2 d, 1H, H-6), 7.39-7.18 (a series of multiplets, 5H, OPh), 6.06, 6.05 (2 s, 1H, H-1′), 5.88-5.84 (2 d, 1H, H-5), 4.61-4.36 (m, 2H, H-5′ & H-5″), 4.33-4.28 (m, 1H, H-α-asp), 4.12-4.08 (m, 1H, H-4′), 3.79-3.76 (2 d, 1H, H-3′), 3.70, 3.65, 3.63, 3.60 (4 s, 6H, OCH₃-asp), 2.86-2.72 (m, 2H, H-β-asp), 1.10, 1.08 (2 s, 3H, —CH₃-2′).

¹³C NMR (125 MHz, MeOD) δ: 174.4 (d, ³J_{CP}=4.44 Hz, —CO-α), 174.1 (d, ³J_{CP}=5.53 Hz, —CO-α), 173.2, 173.0 (—CO-β), 168.2 (C-4), 159.5 (C-2), 153.0, 152.9 (phenyl C), 143.1, 143.0 (C-6), 131.8 (phenyl C), 127.2 (phenyl C), 122.3-122.2 (phenyl C), 97.3 (C-5), 94.7 (C-1′), 82.2-82.1 (C-4′), 80.6, 80.5 (C-2′), 74.9, 74.7 (C-3′), 67.2 (d, ³J_{CP}=4.69 Hz, C-5′), 66.9 (d, ³J_{CP}=4.69 Hz, C-5′), 54.0, 53.9 (OCH₃-asp), 53.6, 53.5 (C-α-asp), 53.3 (OCH₃-asp), 40.1-40.0 (C-β-asp), 21.2 (CH₃-2′).

³¹P NMR (202 MHz, MeOD) δ: 3.65 and 3.52.

HRMS (ESI+) calcd for C₂₂H₃₀N₄O₁₁P [M+H]⁺ 557.1643. found 557.1642.

### Example 27

### 2′-C-methylcytidine-5′-[phenyl-(α-methoxy-β-benzyloxy-aspartyl)]phosphate (16b)

This compound was prepared from 14b.

Yield: 71%; R_{f}=0.22 (CH₂Cl₂/MeOH, 9.2:0.8)

¹H NMR (500 MHz, MeOD): δ=7.84-7.81 (2 d, 1H, H-6), 7.38-7.16 (a series of multiplets, 10H, OPh & CH₂Ph), 6.03, 6.02 (2 s, 1H, H-1′), 5.98-5.92 (2 d, 1H, H-5), 5.08-5.05 (CH₂Ph), 4.60-4.31 (m, 3H, H-5′, H-5″ & H-α-asp), 4.15-4.08 (m, 1H, H-4′), 3.81-3.78 (2 d, 1H, H-3′), 3.65, 3.60 (2 s, 3H, OCH₃-asp), 2.91-2.76 (m, 2H, H-β-asp), 1.13, 1.12 (2 s, 3H, —CH₃-2′).

¹³C NMR (125 MHz, MeOD) δ: 174.3 (d, ³J_{CP}=4.86 Hz, —CO-α), 174.1 (d, ³J_{CP}=5.53 Hz, —CO-α), 172.5, 172.4 (—CO-β), 165.7 (C-4), 156.0 (C-2), 152.9, 152.8 (phenyl C), 144.3, 144.2 (C-6), 138.0 (phenyl C), 131.8, 130.4, 130.2, 130.1, 127.2, 122.3, 122.2 (phenyl C), 97.1 (C-5), 94.8 (C-1′), 82.5-82.3 (C-4′), 80.6 (C-2′), 74.7, 74.5 (C-3′), 68.6 (CH₂Ph), 67.1, 66.8 (C-5′), 54.0, 53.9 (OCH₃-asp), 53.6, 53.5 (C-α-asp), 53.3 (OCH₃-asp), 40.4-40.2 (C-β-asp), 21.1 (CH₃-2′).

³¹P NMR (202 MHz, MeOD) δ: 3.66 and 3.51.

HRMS (ESI−) calcd for C₂₈H₃₄N₄O₁₁P [M−H]⁻ 631.1810. found 631.1801.

### Example 28

### 2′-C-Methylcytidine-5′-[1-phenyl-bis(isopropyl-aspartyl)]phosphate (16c)

This compound was prepared from 14c.

Yield=42%; R_{f}=0.3 (CH₂Cl₂/MeOH, 9.5:0.5)

¹H NMR (500 MHz, MeOD) δ: 7.84-7.81 (2 d, 1H, H-6), 7.39-7.19 (a series of multiplets, 5H, OPh), 6.03, 6.01 (2 s, 1H, H-1′), 5.97-5.93 (2 d, 1H, H-5), 4.99-4.90 (m, 2H, —CH(CH₃)₂), 4.63-4.37 (m, 2H, H-5′ & H-5″), 4.26-4.21 (m, 1H, H-α-Asp), 4.15-4.10 (m, 1H, H-4′), 3.80-3.78 (d, 1H, H-3′), 2.78-2.67 (m, 2H, H-β-Asp), 1.24-1.19 (m, 12H, —CH(CH₃)₂), 1.13 and 1.12 (2 s, 3H, —CH₃-2′). ¹³C NMR (125 MHz, MeOD) δ: 172.6, 172.3, 171.4, 171.3 (—CO-asp), 164.9 (C-4), 155.1 (C-2), 152.1, 152.0 (phenyl C), 143.5 (C-6), 130.9 (phenyl C), 126.3 (phenyl C), 121.4, 121.3 (phenyl C), 96.1 (C-5), 93.9 (C-1′), 81.6, 81.5 (C-4′), 79.7, 79.6 (C-2′), 73.9, 73.7 (C-3′), 70.8, 70.6, 69.8 (CH(CH₃)₂), 66.3, 66.1 (C-5′), 52.9, 52.8 (C-α-Asp), 39.9, 39.7 (C-β-Asp), 22.0-21.9 (—CH(CH₃)₂), 20.3 (2′-CH₃).

³¹P NMR (202 MHz, MeOD) δ: 3.8 and 3.5.

HRMS (ESI−) calcd for C₂₆H₃₆N₄O₁₁P [M−H]⁻ 611.2123. found 611.2126.

### Example 29

### 2′-C-methyluridine-5′-[phenyl-bis(methoxy-aspartyl)]phosphate (17a)

This compound was prepared from 15a.

Yield: 78%; R_{f}=0.16 (CH₂Cl₂/MeOH, 9.5:0.5)

¹H NMR (500 MHz, MeOD): δ=7.69-7.67 (2 d, 1H, H-6), 7.37-7.20 (a series of multiplets, 5H, OPh), 5.98, 5.97 (2 s, 1H, H-1′), 5.65-5.59 (2 d, 1H, H-5), 4.62-4.36 (m, 2H, H-5′ & H-5″), 4.34-4.28 (m, 1H, H-α-asp), 4.12-4.09 (m, 1H, H-4′), 3.85-3.80 (2 d, 1H, H-3′), 3.70, 3.65, 3.63, 3.60 (4 s, 6H, OCH₃-asp), 2.85-2.72 (m, 2H, H-β-asp), 1.16, 1.14 (2 s, 3H, —CH₃-2′).

¹³C NMR (125 MHz, MeOD) δ: 173.5 (d, ³J_{CP}=4.77 Hz, —CO-α), 173.3 (d, ³J_{CP}=5.16 Hz, —CO-α), 172.3, 172.2 (—CO-β), 165.9 (C-4), 152.3 (C-2), 152.1 (phenyl C), 142.0, 141.9 (C-6), 130.9 (phenyl C), 126.3 (phenyl C), 121.4-121.3 (phenyl C), 102.8 (C-5), 93.5, 93.4 (C-1′), 81.6, 81.5 (C-4′), 79.6 (C-2′), 73.9, 73.7 (C-3′), 66.3 (d, ²J_{CP}=5.00 Hz, C-5′), 66.0 (d, ²J_{CP}=4.80 Hz, C-5′), 53.1 (OCH₃-asp), 52.7, 52.6 (C-α-asp), 52.5, 52.4 (OCH₃-asp), 39.3-39.1 (C-β-asp), 20.2 (CH₃-2′).

³¹P NMR (202 MHz, MeOD) δ: 3.68 and 3.60.

HRMS (ESI+) calcd for C₂₂H₂₉N₃O₁₂P [M+H]⁺ 558.1483. found 558.1487.

### Example 30

### 2′-C-methyluridine-5′-[phenyl-(α-methoxy-β-benzyloxy-aspartyl)]phosphate (17b)

This compound was prepared from 15b.

Yield: 63%; R_{f}=0.33 (CH₂Cl₂/MeOH, 9.5:0.5);

¹H NMR (500 MHz, MeOD): δ=7.67-7.65 (2 d, 1H, H-6), 7.36-7.15 (a series of multiplets, 10H, OPh & CH₂Ph),5.97, 5.96 (2 s, 1H, H-1′), 5.64-5.58 (2 d, 1H, H-5), 5.08-5.05 (CH₂Ph), 4.59-4.30 (m, 3H, H-5′, H-5″ & H-α-asp), 4.11-4.07 (m, 1H, H-4′), 3.83-3.78 (2 d, 1H, H-3′), 3.63, 3.59 (2 s, 3H, OCH₃-asp), 2.98-2.74 (m, 2H, H-β-asp), 1.15, 1.12 (2 s, 3H, —CH₃-2′).

¹³C NMR (125 MHz, MeOD) δ: 174.3 (d, ³J_{CP}=4.88 Hz, —CO-α), 174.1 (d, ³J_{CP}=5.65 Hz, —CO-α), 172.5, 172.4 (—CO-β), 166.7 (C-4), 153.1 (C-2), 152.9 (phenyl C), 142.8, 142.7 (C-6), 138.1, 138.0 (CH₂Ph), 131.8, 131.7, 130.4, 130.2, 127.2, 122.2, 122.1 (phenyl C), 103.7, 103.6 (C-5), 94.3, 94.2 (C-1′), 82.4, 82.3 (C-4′), 80.5, 80.4 (C-2′), 74.8, 74.6 (C-3′), 68.6 (CH₂Ph), 67.2 (d, ²J_{CP}=5.33 Hz, C-5′), 66.8 (d, ²J_{CP}=5.06 Hz, C-5′), 53.9 (OCH₃-asp), 53.6, 53.5 (C-α-asp), 40.4-40.2 (C-β-asp), 21.1 (CH₃-2′).

³¹P NMR (202 MHz, MeOD) δ: 3.66 and 3.53.

HRMS (ESI−) calcd for C₂₈H₃₁N₃O₁₂P [M−H]⁻ 632.1651. found 632.1650.

### Example 31

### 2′-C-Methyl-uridine-5′-[1-phenyl-bis(isopropyl-aspartyl)]phosphate (17c)

This compound was prepared from 15c

Yield=86%, R_{f}=0.44 (CH₂Cl₂/MeOH, 9.5:0.5)

¹H NMR (500 MHz, MeOD) δ: 7.68-7.65 (2 d, 1H, H-6), 7.38-7.18 (a series of multiplets, 5H, OPh), 5.98, 5.97 (2 s, 1H, H-1′), 5.65-5.60 (2 d, 1H, H-5), 5.03-4.83 (m, 2H, —CH(CH₃)₂), 4.64-4.40 (m, 2H, H-5′ & H-5″), 4.25-4.21 (m, 1H, —H-α-asp), 4.14-4.08 (m, 1H, H-4′), 3.84-3.82 (2 d, 1H, H-3′), 2.78-2.63 (m, 2H, —H-β-asp), 1.24-1.18 (m, 12H, —CH(CH₃)₂), 1.16, 1.14 (2 s, 3H, —CH₃-2′).

¹³C NMR (125 MHz, MeOD) δ: 172.5 (d, ³J_{CP}=5.07 Hz, —CO-α), 172.3 (d, ³J_{CP}=5.92 Hz, —CO-α), 171.3 (—CO-β), 165.7 (C-4), 152.2 (C-2), 152.1, 152.0 (phenyl C), 141.9, 141.8 (C-6), 130.9 (phenyl C), 126.3 (phenyl C), 121.3 (phenyl C), 102.9, 102.8 (C-5), 93.5, 93.3 (C-1′), 81.5, 81.4 (C-4′), 79.6, 79.5 (C-2′), 73.9, 73.7 (C-3′), 70.7, 69.8, 69.7 —CH(CH₃)₂), 66.4 (d, ²J_{CP}=4.52 Hz, C-5′), 65.9 (d, ²J_{CP}=4.73 Hz, C-5′), 52.8, 52.7 (—C-α-asp), 39.9-39.7 (—H-β-asp), 22.0-21.9 (—CH(CH₃)₂), 20.2 (2′-CH₃).

³¹P NMR (202 MHz, MeOD) δ: 3.82 and 3.59.

HRMS (ESI−) calcd for C₂₆H₃₅N₃O₁₂P [M−H]⁻ 612.1964. found 612.1964.

### Example 32

### 2′-C-Methyl-uridine-5′-[phenyl-bis(n-butyl-aspartyl)]phosphate (17d)

This compound was prepared from 15d.

Yield=80%; R_{f}=0.2 (CH₂Cl₂/MeOH, 9.5:0.5)

¹H NMR (500 MHz, MeOD) δ: 7.68-7.66 (2 d, 1H, H-6), 7.38-7.18 (a series of multiplets, 5H, OPh), 5.98, 5.97 (2 s, 1H, H-1′), 5.65-5.63 (2 d, 1H, H-5), 4.62-4.37 (m, 2H, H-5′ & H-5″), 4.32-4.26 (m, 1H, H-α-Asp), 4.16-3.98 (m, 5H, H-4′ & —OCH₂(CH₂)₂CH₃), 3.84-3.80 (2 d, 1H, H-3′), 2.84-2.69 (m, 2H, H-β-Asp), 1.62-1.53 (m, 4H, —OCH₂CH₂CH₂CH₃), 1.40-1.31 (m, 4H, —O(CH₂)₂CH₂CH₃), 1.16 and 1.13 (2 s, 3H, —CH₃-2′), 0.93-0.90 (m, 6H, —O(CH₂)₃CH₃).

¹³C NMR (125 MHz, MeOD) δ: 174.0 (d, ³J_{CP}=4.89 Hz, —CO-α), 173.7 (d, ³J_{CP}=5.91 Hz, —CO-α), 172.7 (—CO-β), 166.7 (C-4), 153.1 (C-2), 153.0, 152.9 (phenyl C), 142.8, 142.7 (C-6), 131.8 (phenyl C), 127.1 (phenyl C), 122.2, 122.1 (phenyl C), 103.7 (C-5), 94.3, 94.2 (C-1′), 82.4, 82.3 (C-4′), 80.5, 80.4 (C-2′), 74.8, 74.6 (C-3′), 67.5-66.7 (C-5′ & —OCH₂(CH₂)₂CH₃), 53.6, 53.5 (C-α-asp), 40.4-40.2 (C-β-asp), 32.5 (—OCH₂CH₂CH₂CH₃), 21.1-20.9 (CH₃-2′ & —O(CH₂)₂CH₂CH₃), 14.9 (—O(CH₂)₃CH₃).

³¹P NMR (202 MHz, MeOD) δ: 3.73 and 3.57.

HRMS (ESI+) calcd for C₂₈H₃₉N₃O₁₂P [M+H]⁺ 642.2422. found 642.2429.

### Example 33

### 2′-C-Methyl-uridine-5′-[phenyl-bis(amyl-aspartyl)]phosphate (17e)

This compound was prepared from 15e.

Yield=60%; R_{f}=0.4 (CH₂Cl₂/MeOH, 9.5:0.5)

¹H NMR (600 MHz, MeOD) δ: 7.68-7.66 (2 d, 1H, H-6), 7.38-7.18 (a series of multiplets, 5H, OPh), 5.98, 5.97 (2 s, 1H, H-1′), 5.65-5.61 (2 d, 1H, H-5), 4.62-4.38 (m, 2H, H-5′ & H-5″), 4.31-4.26 (m, 1H, H-α-Asp), 4.15-3.99 (m, 5H, H-4′ & —OCH₂(CH₂)₃CH₃), 3.83-3.80 (d, 1H, H-3′), 2.84-2.70 (m, 2H, H-β-Asp), 1.61-1.57 (m, 4H, —OCH₂CH₂(CH₂)₂CH₃), 1.35-1.27 (m, 8H, —O(CH₂)₂(CH₂)₂CH₃), 1.16 and 1.13 (2 s, 3H, —CH₃-2′), 0.91-0.88 (m, 6H, —O(CH₂)₄CH₃).

¹³C NMR (150 MHz, MeOD) δ: 173.9 (d, ³J_{CP}=4.70 Hz, —CO-α), 173.7 (d, ³J_{CP}=5.75 Hz, —CO-α), 172.7 (—CO-β), 166.6 (C-4), 153.1 (C-2), 153.0, 152.9 (phenyl C), 142.8, 142.7 (C-6), 131.8, 131.7 (phenyl C), 127.1 (phenyl C), 122.2 (phenyl C), 103.7 (C-5), 94.2 (C-1′), 82.4, 82.3 (C-4′), 80.5, 80.4 (C-2′), 74.8, 74.5 (C-3′), 67.7 (—OCH₂(CH₂)₃CH₃), 67.2 (d, ²J_{CP}=4.35 Hz, C-5′), 67.1, 67.0 (—OCH₂(CH₂)₃CH₃), 66.8 (d, ²J_{CP}=3.87 Hz, C-5′), 53.6, 53.5 (C-α-asp), 40.4-40.2 (C-β-asp), 30.2-29.9 (—OCH₂(CH₂)₂CH₂CH₃), 24.2 (—O(CH₂)₃CH₂CH₃) 21.1, 21.0 (CH₃-2′), 15.2 (—O(CH₂)₄CH₃).

³¹P NMR (202 MHz, MeOD) δ: 3.74 and 3.57.

HRMS (ESI+) calcd for C₃₀H₄₅N₃O₁₂P [M+H]⁺ 670.2735. found 670.2736.

### Example 34

### 2′-C-Methyl-uridine-5′-[phenyl-bis(isoamyl-aspartyl)]phosphate (17f)

This compound was prepared from 15f.

Yield=86%; R_{f}=0.25 (EtOAc/Hexane, 9.0:1.0)

¹H NMR (600 MHz, MeOD) δ: 7.68-7.66 (2 d, 1H, H-6), 7.38-7.18 (a series of multiplets, 5H, OPh), 5.98, 5.97 (2 s, 1H, H-1′), 5.65-5.61 (2 d, 1H, H-5), 4.62-4.38 (m, 2H, H-5′ & H-5″), 4.31-4.25 (m, 1H, H-α-Asp), 4.19-4.02 (m, 5H, H-4′ & —OCH₂CH₂CH(CH₃)₂), 3.84-3.80 (d, 1H, H-3′), 2.83-2.69 (m, 2H, H-β-Asp), 1.68-1.62 (m, 2H, —OCH₂CH₂CH(CH₃)₂), 1.52-1.46 (m, 4H, —OCH₂CH₂CH(CH₃)₂), 1.16, 1.13 (2 s, 3H, —CH₃-2′), 0.91-0.90 (m, 12H, —OCH₂CH₂CH(CH₃)₂).

¹³C NMR (150 MHz, MeOD) δ: 173.9 (d, ³J_{CP}=4.84 Hz, —CO-α), 173.7 (d, ³J_{CP}=5.68 Hz, —CO-α), 172.7, 172.6 (—CO-β), 166.8, 166.6 (C-4), 153.1 (C-2), 153.0, 152.9 (phenyl C), 142.8, 142.7 (C-6), 131.8, 131.6 (phenyl C), 127.1 (phenyl C), 122.2, 122.1 (phenyl C), 103.7 (C-5), 94.3, 94.2 (C-1′), 82.4, 82.3 (C-4′), 80.5, 80.4 (C-2′), 74.8, 74.5 (C-3′), 67.2 (d, ²J_{CP}=4.63 Hz, C-5′), 66.8 (d, ²J_{CP}=4.32 Hz, C-5′), 66.2, 65.6, 65.5 (—OCH₂CH₂CH(CH₃)₂), 53.6, 53.5 (C-α-asp), 40.4-40.2 (C-β-asp), 39.2, 39.1 (—OCH₂CH₂CH(CH₃)₂), 27.0, 26.9 (—OCH₂CH₂CH(CH₃)₂), 23.7, 23.6 (—OCH₂CH₂CH(CH₃)₂), 21.1 (CH₃-2′).

³¹P NMR (202 MHz, MeOD) δ: 3.72 and 3.56.

HRMS (ESI+) calcd for C₃₀H₄₅N₃O₁₂P [M−H]⁻ 670.2735. found 670.2741.

### Examples 35-37

### General Procedure for N-Cbz Deprotection

To a solution of Cbz-protected phosphoramidate in EtOH (5 mL/mmol) was added 10% Pd/C (10-15 wt. %) at room temperature. The mixture was stirred under H₂ for 3 h. The suspension was filtered and washed with methanol. The filtrate was evaporated to dryness and purified on silica gel column chromatography eluting with MeOH/CH₂Cl₂ in different proportion (generally 2-5% methanol in CH₂Cl₂) to obtain the required compound as white solid. Over all yield of the reaction is in the range of 57-84%.

The following compounds were made according to this procedure

### Example 35

### 2′-C-Methylcytidine-5′-[phenyl-bis(n-butyl-aspartyl)]phosphate (16d)

This compound was made from 14d.

Yield=57%; R_{f}=0.22 (CH₂Cl₂/MeOH, 9.0:1.0)

¹H NMR (500 MHz, MeOD) δ: 7.69-7.67 (2 d, 1H, H-6), 7.39-7.18 (a series of multiplets, 5H, OPh), 6.05, 6.04 (2 s, 1H, H-1′), 5.84-5.82 (2 d, 1H, H-5), 4.62-4.36 (m, 2H, H-5′ & H-5″), 4.31-4.26 (m, 1H, H-α-Asp), 4.17-3.98 (m, 5H, H-4′ & —OCH₂(CH₂)₂CH₃), 3.76-3.74 (d, 1H, H-3′), 2.84-2.72 (m, 2H, H-β-Asp), 1.63-1.53 (m, 4H, —OCH₂CH₂CH₂CH₃), 1.41-1.29 (m, 4H, —O(CH₂)₂CH₂CH₃) 1.10 and 1.08 (2 s, 3H, —CH₃-2′), 0.93-0.89 (m, 6H, —O(CH₂)₃CH₃).

¹³C NMR (125 MHz, MeOD) δ: 174.0 (d, ³J_{CP}=5.13 Hz, —CO-α), 173.7 (d, ³J_{CP}=5.47 Hz, —CO-α), 172.8, 172.7 (—CO-β), 168.3 (C-4), 159.3 (C-2), 153.0 (phenyl C), 143.0 (C-6), 131.8 (phenyl C), 127.1 (phenyl C), 122.3, 122.2 (phenyl C), 97.1 (C-5), 94.8 (C-1′), 82.2 (C-4′), 80.5, 80.4 (C-2′), 75.0, 74.7 (C-3′), 67.5-66.8 (C-5′ & —OCH₂(CH₂)₂CH₃), 53.7, 53.6 (C-α-asp), 40.5-40.3 (C-β-asp), 32.6 (—OCH₂CH₂CH₂CH₃), 21.1-20.9 (CH₃-2′ & —O(CH₂)₂CH₂CH₃), 14.9 (—O(CH₂)₃CH₃).

³¹P NMR (202 MHz, MeOD) δ: 3.71 and 3.48.

HRMS (ESI−) calcd for C₂₈H₄₀N₄O₁₁P [M−H]⁻ 639.2436. found 639.2440.

### Example 36

### 2′-C-Methyl-cytidine-5′-[phenyl-bis(amyl-aspartyl)]phosphate (16e)

This compound was prepared from 14e.

Yield=70%; R_{f}=0.57 (CH₂Cl₂/MeOH, 9.0:1.0)

¹H NMR (500 MHz, MeOD) δ: 7.69-7.66 (2 d, 1H, H-6), 7.39-7.18 (a series of multiplets, 5H, OPh), 6.06, 6.04 (2 s, 1H, H-1′), 5.87-5.82 (2 d, 1H, H-5), 4.61-4.37 (m, 2H, H-5′ & H-5″), 4.32-4.27 (m, 1H, H-α-Asp), 4.14-3.98 (m, 5H, H-4′ & —OCH₂(CH₂)₃CH₃), 3.77-3.74 (d, 1H, H-3′), 2.85-2.72 (m, 2H, H-β-Asp), 1.63-1.57 (m, 4H, —OCH₂CH₂(CH₂)₂CH₃), 1.34-1.29 (m, 8H, —O(CH₂)₂(CH₂)₂CH₃), 1.10 and 1.08 (2 s, 3H, —CH₃-2′), 0.91-0.88 (m, 6H, —O(CH₂)₄CH₃).

¹³C NMR (125 MHz, MeOD) δ: 173.9 (d, ³J_{CP}=5.21 Hz, —CO-α), 173.7 (d, ³J_{CP}=5.86 Hz, —CO-α), 172.8, 172.7 (—CO-β), 168.2 (C-4), 159.3 (C-2), 153.0, 152.9 (phenyl C), 143.0, 142.9 (C-6), 131.7 (phenyl C), 127.1 (phenyl C), 122.3, 122.2 (phenyl C), 97.1 (C-5), 94.9, 94.7 (C-1′), 82.2, 82.1 (C-4′), 80.5, 80.4 (C-2′), 74.9, 74.7 (C-3′), 67.8-66.9 (C-5′ & —OCH₂(CH₂)₃CH₃), 53.7, 53.6 (C-α-asp), 40.5-40.3 (C-β-asp), 30.2-29.9 (—OCH₂(CH₂)₂CH₂CH₃), 24.2 (—O(CH₂)₃CH₂CH₃) 21.2 (CH₃-2′), 15.2 (—O(CH₂)₄CH₃).

³¹P NMR (202 MHz, MeOD) δ: 3.72 and 3.48.

HRMS (ESI+) calcd for C₃₀H₄₆N₄O₁₁P [M+H]⁺ 669.2895. found 669.2894.

### Example 37

### 2′-C-Methylcytidine-5′-[phenyl-bis(isoamyl-aspartyl)]phosphate 16f

This compound was prepared from 14f.

Yield=67%; R_{f}=0.12 (CH₂Cl₂/MeOH, 9.5:0.5)

¹H NMR (500 MHz, MeOD) δ: 7.69-7.67 (2 d, 1H, H-6), 7.39-7.18 (a series of multiplets, 5H, OPh), 6.05, 6.04 (2 s, 1H, H-1′), 5.85-5.82 (2 d, 1H, H-5), 4.62-4.36 (m, 2H, H-5′ & H-5″), 4.31-4.26 (m, 1H, H-α-Asp), 4.20-4.01 (m, 5H, H-4′ & —OCH₂CH₂CH(CH₃)₂), 3.77-3.73 (d, 1H, H-3′), 2.84-2.72 (m, 2H, H-β-Asp), 1.69-1.61 (m, 2H, —OCH₂CH₂CH(CH₃)₂), 1.53-1.45 (m, 4H, —OCH₂CH₂CH(CH₃)₂), 1.10, 1.08 (2 s, 3H, —CH₃-2′), 0.90-0.89 (m, 12H, —OCH₂CH₂CH(CH₃)₂).

¹³C NMR (125 MHz, MeOD) δ: 173.9 (d, ³J_{CP}=4.94 Hz, —CO-α), 173.7 (d, ³J_{CP}=5.88 Hz, —CO-α), 172.7, 172.6 (—CO-β), 168.2 (C-4), 159.3 (C-2), 153.0, 152.9 (phenyl C), 143.0 (C-6), 131.7 (phenyl C), 127.1 (phenyl C), 122.3, 122.2 (phenyl C), 97.1 (C-5), 94.8 (C-1′), 82.2 (C-4′), 80.5, 80.4 (C-2′), 74.9, 74.7 (C-3′), 67.3 (d, ²J_{CP}=4.97 Hz, C-5′), 66.9 (d, ²J_{CP}=4.81 Hz, C-5′), 66.2, 65.6, 65.5 (—OCH₂CH₂CH(CH₃)₂), 53.7, 53.5 (C-α-asp), 40.4-40.3 (C-β-asp), 32.2 (—OCH₂CH₂CH(CH₃)₂), 27.0, 26.9 (—OCH₂CH₂CH(CH₃)₂), 23.7, 23.6 (—OCH₂CH₂CH(CH₃)₂), 21.2 (CH₃-2′).

³¹P NMR (202 MHz, MeOD) δ: 3.71 and 3.47.

HRMS (ESI−) calcd for C₃₀H₄₄N₄O₁₁P [M−H]⁻ 667.2750. found 667.2762.

### 4. Synthesis of Phosphoramidate Prodrugs of 2′C-Me-2′-F-Nucleosides

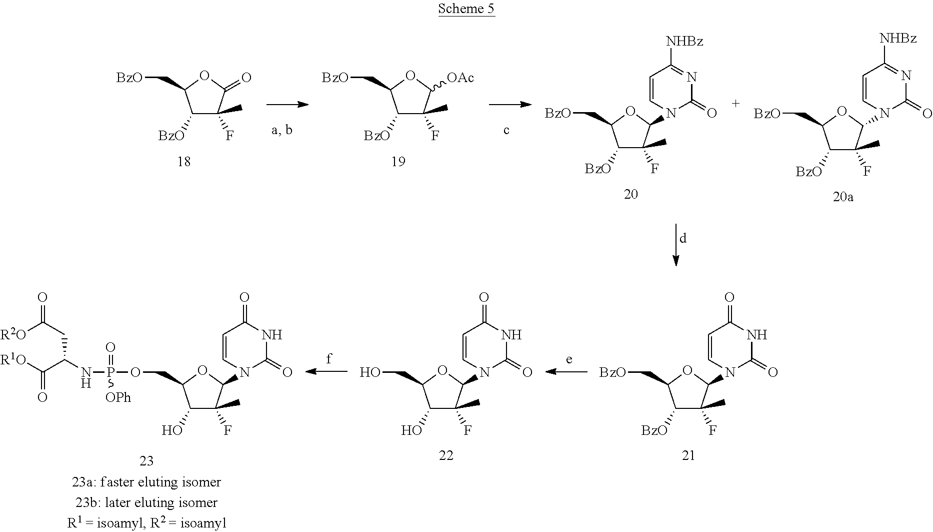

a) Li(O-tBu)₃AlH, THF, −20° C.; b) Ac₂O, DMAP, −20° C.; c) N⁴-benzoylcytosine, N,O-bistrimethylsilylacetamide, SnCl₄, PhCl, 65° C., 16 h; d) 75% aqueous acetic acid, 110° C., 5 h. e) ˜7 N NH₃ in MeOH, rt, 30 h; f) Isoamyl ester of aspartic acid hydrochloride, phenyl dichlorophosphate, N-methylimidazole, dry CH₂Cl₂, −15° C. to rt, overnight, then nucleoside in dry CH₂Cl₂, −5° C. to rt, 24 h.

### Example 38

### 1-O-acetyl-3,5-di-O-benzoyl-2-deoxy-2-fluoro-2-C-methyl-α,β-D-ribofuranose (19)

This compound was synthesized according to a known procedure: J. Org. Chem. 2009, 74, 6819-6824.

Protected lactone 18 (2 g, 5.4 mmol) was dissolved in dry tetrahydrofuran (45 mL) under nitrogen atmosphere and the solution was cooled to −20° C. Lithium tri-tert-butoxyaluminium hydride (1.0 M in THF, 6.5 mL, 6.5 mmol) was added dropwise over 20 min while maintaining the temperature near −20° C. Upon completion of the reaction (˜3 h) based on TLC, that is the formation of lactol (R_{f}=0.36, 2:8 EtOAc/Hexane), DMAP (66 mg, 5.4 mmol) and acetic anhydride (4.7 mL, 49.4 mmol) were added to the reaction mixture at −20° C. and stirred for 1.5 h. The reaction mixture was diluted with ethyl acetate and water. The organic layer was collected and the aqueous layer was extracted three times with ethyl acetate. The combined organic layer was dried over anhydrous MgSO₄, filtered and concentrated under reduced pressure to obtain crude acetate 19 which was purified by flash column chromatography using 0-15% EtOAc in hexane to obtain the pure product as clear oil in 95% yield. R_{f}=0.5 (2:8 EtOAc/Hexane).

¹H NMR (500 MHz, DMSO-d6): δ=8.03-8.01 (m, 4H, Ar—H), 7.99-7.94 (m, 4H, Ar—H), 7.74-7.70 (m, 2H, Ar—H), 7.68-7.65 (m, 2H, Ar—H), 7.59-7.56 (m, 4H, Ar—H), 7.52-7.49 (m, 4H, Ar—H), 6.19 (d, 1H, J=4.33 Hz, H-1a), 6.09 (d, 1H, J=9.65 Hz, H-1 b), 5.62 (dd, J=7.97, 24.50 Hz, 1H, H-3a), 5.62 (dd, J=6.25, 8.59 Hz, 1H, H-3b), 4.75-4.72 (m, 1H, H-4a), 4.67-4.61 (m, 3H, H-4b & H-5a), 4.57-4.41 (m, 2H, H-5b), 2.14 (s, 3H, OAc-a), 1.92 (s, 3H, OAc-b), 1.62 (d, 3H, J=22.96 Hz, CH₃-a), 1.50 (d, 3H, J=23.37 Hz, CH₃-b).

¹³C NMR (125 MHz, DMSO-d6): δ=168.9, 168.3 (CO of —OAc), 165.0, 164.8, 164.7, 164.5 (CO of Bz), 133.6, 133.5, 133.2 (Ar—C), 129.2-128.1 (Ar—C), 100.2, 97.4, 95.3, 93.7 (C-1a, C-1b, C-2a & C-2b), 79.0, 77.9 (C-4a, C-4b), 73.3, 73.2, 72.7, 72.6 (C-3a, C-3b), 63.0, 62.6 (C-5a, C-5b), 20.4-20.0 (CH₃), 15.8, 15.6 (—CH₃).

HRMS (ESI+) calcd for C₂₂H₂₁F₁O₇Na [M+Na]⁺ 439.1164. found 439.1160.

### Example 39

### N⁴-Benzoyl-3′,5′-di-O-benzoyl-2′-deoxy-2′-fluoro-2′-C-methylcytidine (20)

To a suspension of N⁴-benzoylcytosine (1.74 g, 8.0 mmol) in anhydrous chlorobenzene (24 mL), N,O-bis(trimethylsilyl)acetamide (4.5 mL, 18 mmol) was added and the suspension was heated to 80° C. for 2 h. The clear resultant solution was then cooled to room temperature. A solution of acetate sugar 19 (1.68 g, 4.0 mmol) in chlorobenzene (12 mL) was then added to the silylated base. To this, neat tin (IV) chloride (2.4 mL, 20 mmol) was added dropwise and was heated to 65° C. for 16 h. The reaction mixture was cooled to room temperature and diluted with ethyl acetate. Cold saturated sodium bicarbonate solution was added and the white suspension was then filtered through a celite pad. The organic layer was separated and the aqueous layer was extracted with ethyl acetate several times. The combined organic layer was washed with brine, dried over anhydrous MgSO₄, filtered and concentrated under reduced pressure to obtain the crude product (a mixture of a and 3 isomer) which was purified by flash column chromatography eluting with 20-40% EtOAc in hexane to obtain the pure β-isomer (20) in 26% yield. R_{f}=0.34 for β-isomer (1:1 EtOAc/Hexane) and R_{f}=0.2 for α-isomer (20a) (1:1 EtOAc/Hexane).

β isomer (20): ¹H NMR (500 MHz, CDCl₃): δ=8.70 (br s, 1H, NH), 8.10-8.06 (m, 5H, Ar—H), 7.89 (d, J=7.03 Hz, 2H), 7.69-7.61 (m, 3H, Ar—H), 7.55-7.46 (m, 7H, Ar—H), 6.19 (br d, 1H, J=16.59 Hz, H-1′), 5.55 (br dd, J=8.6, 20.7 Hz, 1H, H-3′), 4.88 (dd, J=2.4, 12.7 Hz, 1H, H-5′), 4.72 (m, 1H, H-4′), 4.63 (dd, J=3.27, 12.7 Hz, 1H, H-5″), 1.48 (d, 3H, J=22.4 Hz, —CH₃).

HRMS (ESI+) calcd for C₃₁H₂₇F₁N₃O₇ [M+H]⁺ 572.1827. found 572.1832.

### Example 40

### 3′,5′-Di-O-benzoyl-2′-deoxy-2′-fluoro-2′-C-methyluridine (21)

A suspension of compound 20 (0.58 g, 1.0 mmol) in 75% aqueous acetic acid (30 mL) was heated to 110° C. for 5 h. The clear solution was cooled to room temperature and concentrated to dryness under reduced pressure and coevaporated with methanol/water (1:1) for three times to remove traces of acetic acid. The compound 21 was used as such without further purification for the next step. Yield: 90%, R_{f}=0.45 (EtOAc/Hexane, 1:1)

¹H NMR (300 MHz, CDCl₃+CD₃OD): δ=8.05-7.96 (m, 4H, Ar—H), 7.61-7.40 (m, 7H, Ar—H & H-6), 6.22 (d, 1H, J=19.05 Hz, H-1′), 5.51 (dd, J=9.47, 21.2 Hz, 1H, H-3′), 5.42 (d, 1H, J=8.11 Hz, H-5), 4.84 (dd, J=2.65, 12.7 Hz, 1H, H-5′), 4.60 (m, 1H, H-4′), 4.49 (dd, J=3.45, 12.7 Hz, 1H, H-5″), 1.42 (d, 3H, J=22.4 Hz, —CH₃).

HRMS (ESI+) calcd for C₂₄H₂₁F₁N₂O₇Na [M+Na]⁺ 491.1225. found 491.1229.

### Example 41

### 2′-deoxy-2′-fluoro-2′-C-methyluridine (22)

NH₃ in methanol (˜7 N, 30 mL) was added to compound 6 (0.5 g, 1.0 mmol) and was stirred 30 h at room temperature. The reaction mixture was evaporated with silica gel and chromatographed on a flash silica gel column eluting with CH₂Cl₂/MeOH/NH₃ (9.0:1.0:0.2) to obtain compound 1 as white solid (62%). TLC (CH₂Cl₂/MeOH/NH₃, 9.0:1.0:0.2): R_{f}=0.23.

¹H NMR (600 MHz, CD₃OD): δ=8.07 (d, J=7.89 Hz, 1H, H-6), 6.12 (d, 1H, J=18.53 Hz, H-1′), 5.71 (d, J=7.89 Hz, 1H, H-5), 4.02-3.79 (m, 4H, H-3′, H-4′, H-5′ & H-5″), 1.35 (d, 3H, J=22.3 Hz, CH₃).

¹³C NMR (150 MHz, CD₃OD): δ=165.8 (C-4), 152.3 (C-2), 141.8 (C-6), 102.9 (C-5), 102.0 (d, J=181.0, C-2′), 90.5 (br d, C-1′), 83.3 (C-4′), 72.4 (d, J=18.0, C-3′), 60.0 (C-5′), 16.8 (d, J=25.5, —CH₃).

HRMS (ESI+) calcd for C₁₀H₁₃F₁N₂O₅Na [M+Na]⁺ 283.0701. found 283.0709.

### Example 42

### 2′-Deoxy-2′-fluoro-2′-C-methyl-uridine-5′-[phenyl-bis(isoamyl-aspartyl)]phosphate (23a, faster eluting diastereoisomer)

This compound was synthesized according to the general procedure for the preparation of phosphoramidates (see example 14).

R_{f}=0.45 (MeOH/CH₂Cl₂, 9.5:0.5)

¹H NMR (500 MHz, CDCl₃) δ: 8.56 (1H, NH), 7.36-7.33 (m, 3H, H-6 & OPh), 7.22-7.18 (m, 3H, OPh), 6.18 (d, J=18.84 Hz, 1H, H-1′), 5.62 (d, J=8.04 Hz, 1H, H-5), 4.59-4.54 (m, 2H, H-5′ & H-5″), 4.31-4.05 (m, 7H, H-α-Asp, NH-Asp, H-4′ & —OCH₂CH₂CH(CH₃)₂), 3.92-3.80 (m, 1H, H-3′), 3.64 (br s, 3′-OH), 2.96-2.55 (m, 2H, H-β-Asp), 1.65-1.59 (m, 2H, —OCH₂CH₂CH(CH₃)₂), 1.50-1.48 (m, 4H, —OCH₂CH₂CH(CH₃)₂), 1.36 (d, J=22.4 Hz, 3H, —CH₃-2′), 0.91-0.89 (m, 12H, —OCH₂CH₂CH(CH₃)₂).

¹³C NMR (125 MHz, CDCl₃) δ: 171.8 (d, ³J_{CP}=6.08 Hz, —CO-α), 171.1 (—CO-β), 162.5 (C-4), 150.5 (d, J_{CP}=6.63 Hz, phenyl C), 150.2 (C-2), 139.1 (C-6), 130.1, 125.7, 120.2 (phenyl C), 103.1 (C-5), 100.5 (d, J=182.12 Hz, C-2′), 89.3 (C-1′), 80.1 (C-4′), 71.7 (d, J=17.8 Hz, C-3′), 65.1, 64.1 (—OCH₂CH₂CH(CH₃)₂), 63.9 (C-5′), 51.6 (C-α-Asp), 38.3 (d, J_{CP}=4.12 Hz, C-β-Asp), 37.3, 37.2 (—OCH₂CH₂CH(CH₃)₂), 25.2, 25.1 (—OCH₂CH₂CH(CH₃)₂), 22.6, 22.5 (—OCH₂CH₂CH(CH₃)₂), 16.7 (d, J=25.5 Hz, —CH₃-2′).

³¹P NMR (202 MHz, CDCl₃): δ=4.41.

HRMS (ESI+) calcd for C₃₀H₄₂F₁N₃O₁₁P [M−H]⁻ 670.2746. found 670.2545.

### Example 43

### 2′-Deoxy-2′-fluoro-2′-C-methyl-uridine-5′-[phenyl-bis(isoamyl-aspartyl)]phosphate (23b, later eluting diastereoisomer)

This compound was synthesized according to the general procedure for the preparation of phosphoramidates (see example 14).

R_{f}=0.40 (MeOH/CH₂Cl₂, 9.5:0.5)

¹H NMR (500 MHz, CDCl₃) δ: 8.61 (1H, NH), 7.46 (d, J=8.23 Hz, 1H, H-6), 7.37-7.34 (m, 2H, OPh), 7.24-7.18 (m, 3H, OPh), 6.18 (d, J=17.78 Hz, 1H, H-1′), 5.66 (d, J=8.23 Hz, 1H, H-5), 4.57-4.46 (m, 2H, H-5′ & H-5″), 4.33-4.06 (m, 7H, H-α-Asp, NH-Asp, H-4′ & —OCH₂CH₂CH(CH₃)₂), 3.98-3.81 (m, 1H, H-3′), 3.68 (br s, 3′-OH), 2.92-2.67 (m, 2H, H-β-Asp), 1.66-1.59 (m, 2H, —OCH₂CH₂CH(CH₃)₂), 1.52-1.46 (m, 4H, —OCH₂CH₂CH(CH₃)₂), 1.42 (d, J=22.4 Hz, 3H, —CH₃-2′), 0.91-0.89 (m, 12H, —OCH₂CH₂CH(CH₃)₂).

¹³C NMR (125 MHz, CDCl₃) δ: 171.4 (d, ³J_{CP}=5.97 Hz, —CO-α), 170.7 (—CO-β), 162.6 (C-4), 150.6 (d, J_{CP}=6.01 Hz, phenyl C), 150.2 (C-2), 139.4 (C-6), 130.1, 125.5, 120.2, 119.9 (phenyl C), 103.1 (C-5), 100.5 (d, J=181.6 Hz, C-2′), 89.2 (C-1′), 80.0 (C-4′), 71.9 (d, J=18.5 Hz, C-3′), 65.1, 64.2 (—OCH₂CH₂CH(CH₃)₂), 63.9 (C-5′), 51.4 (C-α-Asp), 38.6 (d, J_{CP}=3.96 Hz, C-β-Asp), 37.3, 37.2 (—OCH₂CH₂CH(CH₃)₂), 25.1 (—OCH₂CH₂CH(CH₃)₂), 22.5, 22.4 (—OCH₂CH₂CH(CH₃)₂), 16.7 (d, J=25.26 Hz, —CH₃-2′).

³¹P NMR (202 MHz, CDCl₃): δ=3.50.

HRMS (ESI+) calcd for C₃₀H₄₂F₁N₃O₁₁P [M−H]⁻ 670.2746. found 670.2548.

### 5. Synthesis of Phosphoramidate Prodrugs of Gemcitabine

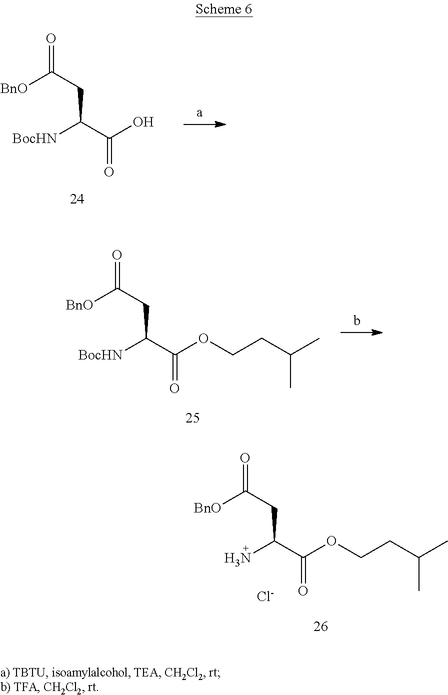 [SMILES: CC(C)(C)OC(=O)N[C@@H](CC(=O)OCc1ccccc1)C(=O)O ; CC(C)CCOC(=O)[C@H](CC(=O)OCc1ccccc1)NC(=O)OC(C)(C)C ; CC(C)CCOC(=O)[C@@H](N)CC(=O)OCc1ccccc1 ; [Cl-]]

### Example 44

### Synthesis of Boc-L-Asp-(OBn)-O-isoamyl (25)

To a suspension of Boc-Asp(OBn)-OH (1.62 g, 5.0 mmol) in anhydrous dichloromethane (40 ml) at room temperature was added N,N,N′,N′-Tetramethyl-O-(6-chloro-1H-benzotriazol-1-yl)uronium hexafluorophosphate (TBTU, 2.28 g, 5.5 mmol). The resulting mixture was stirred at room temperature for 30 minutes and then isoamyl alcohol (3 ml, 28 mmol) and Et₃N (2 mL, 21 mmol) were added. The mixture was stirred at room temperature for another 4 hours. The solvent was removed under reduced pressure. The residue was dissolved in ethyl acetate (50 ml) and washed with water and brine. The organic layer was dried over MgSO₄ and concentrated under reduced pressure. The crude residue was purified by flash silica gel column chromatography (eluting with EtOAc in cyclohexane 0-20%) to yield the title compound as a colorless oil (1.90 g, 96%).

¹H NMR (300 MHz, CDCl₃): δ=7.36 (m, 5H, Ar—H), 5.50 (d, 1H, —NH), 5.15 (s, 2H, OCH₂), 4.59 (m, 1H, CH), 4.16 (t, J=6.8 Hz, 2H, OCH₂), 3.06 (dd, J=17.2 Hz and J=4.7 Hz, 1H, H-α), 2.88 (dd, J=16.9 Hz and J=4.7 Hz, H-b), 1.62 (m, 1H, CH), 1.47 (m, 2H, CH₂), 1.46 (s, 9H, CH₃), 0.91 (m, 6H, CH₃) ppm.

### Example 45

### Synthesis of L-Asp-(OBn)-O-isoamyl hydrochloride salt (26)

To a solution of Boc-L-Asp-(OBn)-O-isoamyl (1.57 g, 4.0 mmol) in dichloromethane (10 ml), was added trifluoroacetic acid (10 ml) at room temperature. The mixture was stirred at room temperature for 1 hour. After concentration under reduced pressure, the residue was dissolved in dichloromethane (30 ml) and washed with a 5% Na₂CO₃ solution (10 mL). The organic phase was collected and treated with a 1.25 M HCl solution in isopropanol (5 ml). Concentration under reduced pressure yielded the title compound as a white solid (1.25 g, 95%).

¹H NMR (300 MHz, DMSO-d₆): δ=8.76 (s, 3H, NH₃), 7.38 (m, 5H, Ar—H), 5.15 (s, 2H, OCH₂), 4.35 (m, 1H, CH), 4.11 (m, 2H, OCH₂), 3.08 (m, 2H, CH₂), 1.60 (m, 1H, CH), 1.42 (m, 2H, CH₂), 0.85 (m, 6H, CH₃) ppm.

¹³C NMR (75 MHz, DMSO-d₆): δ=169.16, 168.36, 135.66, 128.58, 128.37, 128.27, 66.49, 64.45, 48.56, 36.59, 34.30, 24.32, 22.35, 22.25 ppm.

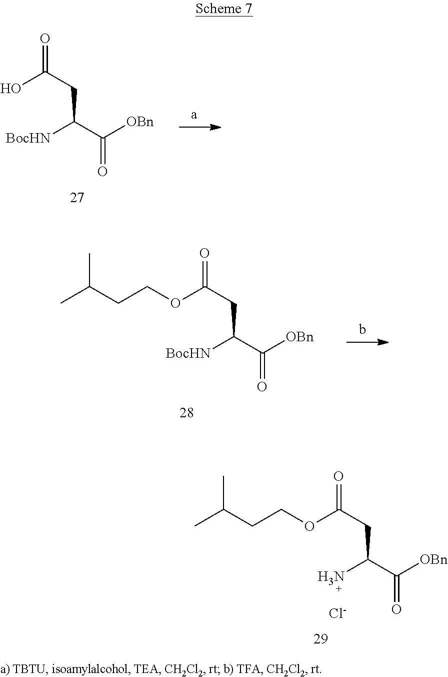 [SMILES: CC(C)(C)OC(=O)N[C@@H](CC(=O)O)C(=O)OCc1ccccc1 ; CC(C)CCOC(=O)C[C@H](NC(=O)OC(C)(C)C)C(=O)OCc1ccccc1 ; CC(C)CCOC(=O)C[C@H](N)C(=O)OCc1ccccc1 ; [Cl-]]

### Example 46

### Synthesis of Boc-L-Asp-(O-Isoamyl)-OBn (28)

The title compound was synthesized from Boc-L-Asp-OBn in 95% yield, according to the procedure mentioned for example 44.

¹H NMR (300 MHz, CDCl₃): δ=7.36 (m, 5H, Ar—H), 5.52 (m, 1H, —NH), 5.20 (s, 2H, OCH₂), 4.63 (m, 1H, CH), 4.09 (t, J=6.9 Hz, 2H, OCH₂), 3.02 (dd, J=17.2 and J=4.8 Hz, 1H, H-α), 2.88 (dd, J=16.9 and J=4.8 Hz, H-b), 1.66 (m, 1H, CH), 1.50 (m, 2H, CH₂), 1.45 (s, 9H, CH₃), 0.92 (d, J=6.6 Hz, 6H, CH₃) ppm.

### Example 47

### Synthesis of Boc-L-Asp-(O-Isoamyl)-OBn (29)

The title compound was synthesized from Boc-L-Asp(O-isoamyl)-OBn in 88% yield, according to the procedure of example 45.

¹H NMR (300 MHz, DMDO-d₆): δ=8.90 (s, 3H, NH₃), 7.39 (m, 5H, Ar—H), 5.20 (s, 2H, OCH₂), 4.39 (m, 1H, CH), 4.03 (t, J=6.8 Hz, 2H, OCH₂), 3.06 (m, 2H, CH₂), 1.58 (m, 1H, CH), 1.42 (m, 2H, CH₂), 0.85 (d, J=6.6 Hz, 6H, CH₃) ppm.

¹³C NMR (75 MHz, DMSO-d₆): δ=169.23, 168.27, 135.17, 128.54, 128.43, 128.14, 67.37, 63.46, 48.62, 36.70, 34.27, 24.54, 22.39, 22.36 ppm.

### Example 48

### 3′-O-(tert-Butoxycarbonyl)gemcitabine

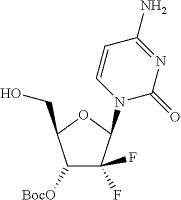

To a stirring mixture of gemcitabine (1.05 g, 4.0 mmol) and Na₂CO₃ (3.12 g, 20.0 mmol) in a mixture of dioxane (40 mL) and water (1 mL) was added di-tert-butyl dicarbonate (DBDC, 873 mg, 4.0 mmol). The resulting mixture was stirred at room temperature for 72 hours. Water (20 ml) was added and the mixture was extracted with dichloromethane (100 mL). The organic extracts were washed with water (20 mL) and brine (20 mL), dried over Na₂SO₄, and concentrated to dryness under reduced pressure. The residue was purified by silicagel flash chromatography (using a mixture of methanol and dichloromethane in a gradient gradually ranging from 0% to 20% methanol) to give the title compound as white solid (1.28 g, 88%).

¹H NMR (300 MHz, CDCl₃) δ: 7.59 (d, J=7.4 Hz, 1H, Ar—H), 7.03 (br., 1H, NH), 6.32 (br., 1H, NH), 6.27 (t, J=9.4 Hz, 1H, H-1′), 5.79 (d, J=7.4 Hz, 1H, Ar—H), 5.79 (m, IH, H-3′), 4.95 (m, 1H, H-4′), 4.03 (m, 1H, H-5′), 3.70 (m, 1H, H-5′) 1.43 (s, 9H, CH₃) ppm.

### Example 49

### 3′-O-(tert-Butoxycarbonyl)gemcitabine-5′-[phenyl-bis(isoamyl-L-aspartyl)]phosphate

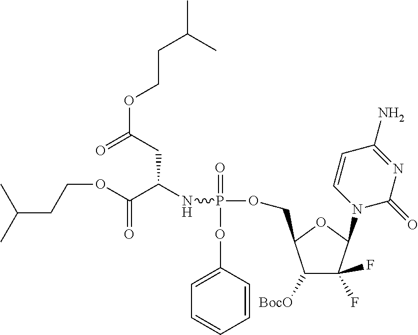

To a mixture of L-aspartic acid diisoamyl-ester hydrochloride (465 mg, 1.5 mmol) in anhydrous CH₂Cl₂ (10 ml) at −40° C. were added dichlorophenyl phosphate (240 μl, 1.5 mmol) and N-methylimidazole (420 μl, 5 mmol), respectively. The mixture was stirred and allowed to warm to room temperature. Stirring was continued for another 12 hours. The mixture was cooled to −40° C., and 3′-O-(tert-Butoxycarbonyl)gemcitabine (181 mg, 0.5 mmol) was added. The mixture was stirred and warmed to room temperature. Stirring was continued till starting material was completely consumed according to TLC analysis. The reaction mixture was then evaporated to dryness under reduced pressure, and the residue was purified by silicagel flash chromatography (using a mixture of methanol and dichloromethane as mobile phase, in a gradient gradually ranging from 0 to 10% methanol) to yield the title compound as a white solid (300 mg, 77%).

¹H NMR (300 MHz, DMSO-d₆) δ: 7.73 (br., 2H, NH₂), 7.35 (m, 1H, Ar—H), 7.30 (m, 2H, Ar—H), 7.18 (m, 3H, Ar—H), 6.25 (m, 1H), 5.78 (m, 1H), 5.39 (m, 1H, OCH), 5.23 (m, 1H), 4.00-4.40 (m, 6H, OCH₂), 3.65 (m, 1H, CH), 2.72 (m, 2H, CH₂), 1.62 (m, 2H, CH₂), 1.45 (s, 9H, CH₃), 1.44 (m, 4H, CH₂), 0.85 (m, 12H, CH₃) ppm.

³¹P NMR (202 MHz, DMSO-d₆) δ: 4.42, 4.36 ppm.

### Example 50

### 3′-O-(tert-Butoxycarbonyl)gemcitabine-5′-[phenyl-bis(ethyl-L-glutamyl)]phosphate

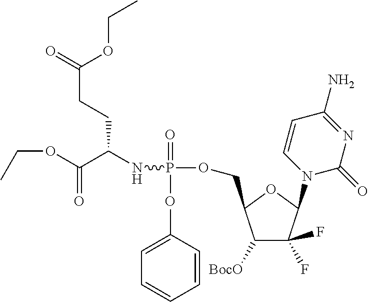

This compound was prepared in 86% yield, using the procedure of example 49.

¹H NMR (300 MHz, DMSO-d₆) δ: 7.40 (m, 1H, Ar—H), 7.27 (m, 2H, Ar—H), 7.15 (m, 3H, Ar—H), 6.70 (br., 2H, NH₂) 6.29 (m, 1H), 5.77 (m, IH), 5.12 (m, 1H), 4.00-4.33 (m, 9H, OCH₂), 2.27 (m, 2H, CH₂), 1.85 (m, 2H, CH₂), 1.42 (s, 9H, CH₃), 1.15 (m, 6H, CH₃) ppm.

³¹P NMR (202 MHz, DMSO-d₆) δ: 3.81, 3.63 ppm.

### Example 51

### 3′-O-(tert-Butoxycarbonyl)gemcitabine-5′-[phenyl-bis(isoamyl-L-glutamyl)]phosphate

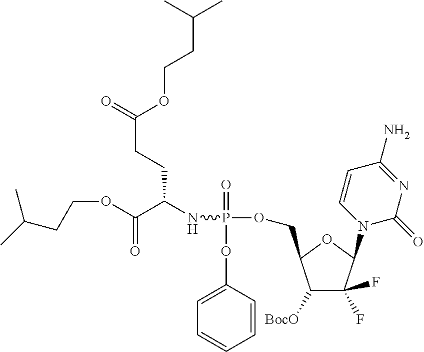

This compound was prepared in 82% yield, using the procedure of example 49.

¹H NMR (300 MHz, CDCl₃) δ: 7.00-7.40 (m, 6H, Ar—H), 6.36 (m, 1H, NH), 5.85 (m, 1H), 5.13 (m, 1H), 4.00-4.50 (m, 9H, OCH & OCH₂), 2.35 (m, 2H, CH₂), 1.98 (m, 2H, CH₂), 1.66 (m, 2H, CH₂), 1.51 (s, 9H, CH₃), 1.50 (m, 4H, CH₂), 0.91 (m, 12H, CH₃) ppm.

³¹P NMR (202 MHz, CDCl₃) δ: 2.91 ppm.

### Example 52

### 3′-O-(tert-Butoxycarbonyl)gemcitabine-5′-[phenyl-(4-benzyl-1-isoamyl-L-aspartyl)]phosphate

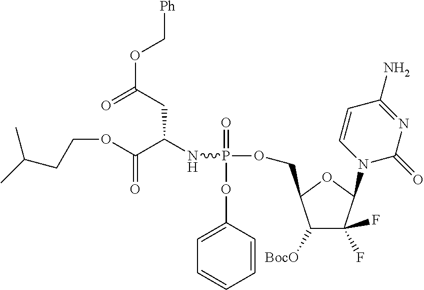

This compound was prepared in 80% yield, using the procedure of example 49.

¹H NMR (300 MHz, CDCl₃) δ: 7.22-7.52 (m, 10H, Ar—H), 6.29 (m, 1H, NH), 6.22 &7.55 (m, 1H, Ar—H), 5.10 (m, 1H, Ar—H), 5.09 & 5.10 (s, OCH₂), 4.00-4.50 (m, 6H, OCH & OCH₂), 2.97 &2.78 (m, 2H, CH₂), 1.61 (m, 1H, CH), 1.51 (s, 7H, CH₃), 1.46 (m, 2H, CH₂), 0.88 (m, 12H, CH₃) ppm.

³¹P NMR (202 MHz, CDCl₃) δ: 3.02, 2.68 ppm.

### Example 53

### 3′-O-(tert-Butoxycarbonyl)gemcitabine-5′-[phenyl-(1-benzyl-4-isoamyl-L-aspartyl)]phosphate

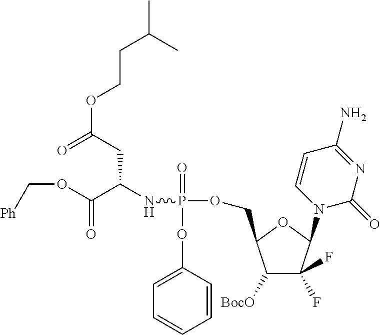

This compound was prepared in 75% yield, using the procedure of example 49.

¹H NMR (300 MHz, CDCl₃) δ: 7.20-7.40 (m, 11H, Ar—H), 6.36 (m, 1H, NH), 5.80 (m, 1H), 5.15 (m, 3H), 4.00-4.50 (m, 7H, OCH & OCH₂), 2.95 & 2.83 (m, 2H, CH₂), 1.44-1.78 (m, 3H, CH₂ & CH), 1.51 (s, 9H, CH₃), 0.88 (m, 6H, CH₃) ppm.

³¹P NMR (202 MHz, CDCl₃) δ: 2.93, 2.53 ppm.

### Example 54

### Gemcitabine-5′-[phenyl-bis(isoamyl-L-aspartyl)]phosphate

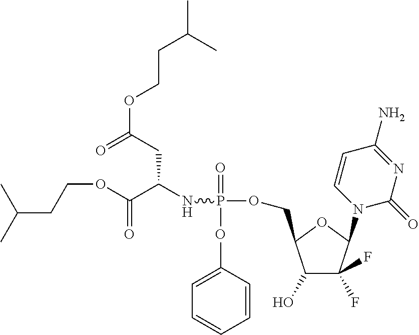

A solution of 3′-O-(tert-Butoxycarbonyl)gemcitabine-5′-[phenyl-bis(isoamyl-aspartyl)]phosphate (250 mg, 0.32 mmol) in TFA/DCM (1/1; 10 ml) was stirred at room temperature for 2 hours. After concentration under the reduced pressure, the residue was purified by flash column chromatography (using a mixture of methanol and dichloromethane as mobile phase, with a gradient ranging from 0-20% methanol) to yield the title compound as a white solid (200 mg, 91%).

¹H NMR (300 MHz, DMSO-d₆) δ: 8.64 & 8.18 (brs, 2H, NH₂), 7.73 (brs, 2H, NH₂), 7.66 (m, 1H, Ar—H), 7.37 (m, 2H, Ar—H), 7.20 (m, 3H, Ar—H), 6.53 (m, 1H, NH), 6.18 (m, 1H), 5.93 (m, 1H, Ar—H), 4.00-4.35 (m, 9H, CHO & OCH₂), 2.63 (m, 2H, CH₂), 1.60 (m, 2H, CH₂), 1.44 (m, 2H, CH₂), 0.85 (m, 12H, CH₃) ppm.

³¹P NMR (202 MHz, DMSO-d₆) δ: 3.64 ppm.

### Example 55

### Gemcitabine-5′-[phenyl-(4-benzyl-1-isoamyl-L-aspartyl)]phosphate

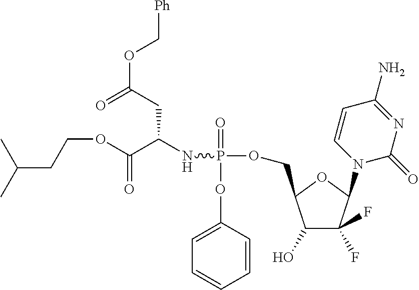

This compound was prepared in 84% yield, according to the procedure of example 54.

¹H NMR (300 MHz, CDCl₃) δ: 8.66 & 8.27 (brs, 2H, NH₂), 7.65 (m, 1H, Ar—H), 7.35 (m, 7H, Ar—H), 7.20 (m, 3H, Ar—H), 6.26 (m, 1H, NH), 6.18 (m, 1H, OCH), 5.95 (m, 1H, Ar—H), 5.06 & 5.04 (s, 2H, OCH₂), 3.80-4.40 (m, 5H, OCH & OCH₂), 2.70 (m, 2H, CH₂), 1.57 (m, 1H, CH), 1.38 (m, 2H, CH₂), 0.82 (m, 6H, CH₃) ppm.

³¹P NMR (202 MHz, CDCl₃) δ: 3.66 ppm.

### Example 56

### Gemcitabine-5′-[phenyl-(1-benzyl-4-isoamyl-L-aspartyl)]phosphate

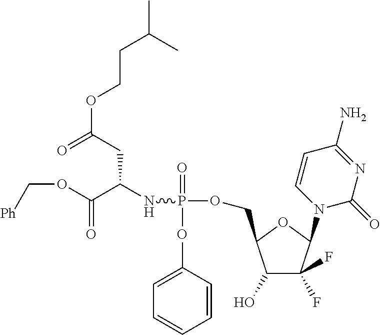

This compound was prepared in 86% yield, according to the procedure of example 54.

¹H NMR (300 MHz, CDCl₃) δ: 8.50 & 8.15 (brs, 2H, NH₂), 7.63 (m, 1H, Ar—H), 7.35 (m, 7H, Ar—H), 7.18 (m, 3H, Ar—H), 6.29 (m, 1H, NH), 6.18 (m, 1H, OCH), 5.90 (m, 1H, Ar—H), 5.10 (m, 2H, OCH₂), 3.80-4.40 (m, 5H, OCH & OCH₂), 2.63 (m, 2H, CH₂), 1.57 (m, 1H, CH), 1.38 (m, 2H, CH₂), 0.83 (m, 6H, CH₃) ppm.

³¹P NMR (202 MHz, CDCl₃) δ: 3.67 ppm.

### Example 57

### Gemcitabine-5′-[phenyl-bis(ethyl-L-glutamyl)]phosphate

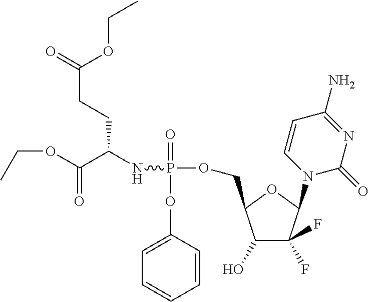

This compound was prepared in 86% yield, according to the procedure of example 54.

¹H NMR (300 MHz, DMSO-d₆) δ: 7.94 & 7.72 (brs, 2H, NH₂), 7.55 (m, 1H, Ar—H), 7.37 (m, 2H, Ar—H), 7.19 (m, 3H, Ar—H), 6.47 (m, 1H, NH), 6.16 (m, 2H), 5.83 (m, 1H), 3.80-4.33 (m, 8H, OCH₂), 2.30 (m, 2H, CH₂), 1.90 & 1.75 (m, 2H, CH₂), 1.15 (m, 6H, CH₃) ppm. ³¹P NMR (202 MHz, DMSO-d₆) δ: 3.98, 3.88 ppm.

### Example 58

### Gemcitabine-5′-[phenyl-bis(isoamyl-L-glutamyl)]phosphate

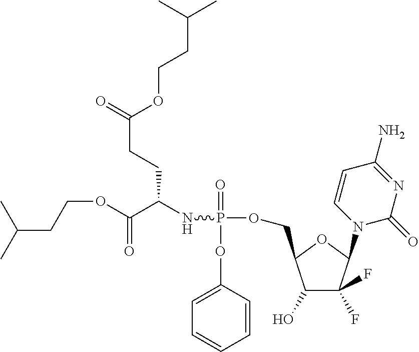

This compound was prepared in 58% yield, according to the procedure of example 54.

¹H NMR (300 MHz, CDCl₃) δ: 7.00-7.40 (m, 6H, Ar—H), 6.10 (m, 2H), 4.00-4.50 (m, 7H, OCH & OCH₂), 2.35 (m, 2H, CH₂), 1.98 (m, 2H, CH₂), 1.63 (m, 2H, CH₂), 1.49 (m, 4H, CH₂), 0.90 (m, 12H, CH₃) ppm.

³¹P NMR (202 MHz, DMSO-d₆) δ: 3.11, 3.02 ppm.

### 6. Synthesis of a Phosporamidate Prodrug of 2′-Deoxy-2′-α-Fluoro-Uridine

### Example 59

### 3′-O-(tert-butoxycarbonyl)-2′-deoxy-2′-fluorouridine

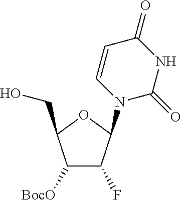

This compound was prepared from 2′-deoxy-2′-α-fluoro-uridine in 73% yield, according to the procedure of example 48.

¹H NMR (300 MHz, CDCl₃) δ: 11.46 (s, 1H, NH), 7.86 (d, J=8.07 Hz, 1H, Ar—H), 5.95 (dd, J=18.4 Hz and J=3.3 Hz, 1H, H-′), 5.68 (d, J=8.04 Hz, 1H, Ar—H), 5.46 (m, 1H), 5.28 (m, 1H), 5.11 (m, 1H), 4.12 (m, 1H), 3.63 (m, 2H), 1.45 (s, 9H, CH₃) ppm.

### Example 60

### 3′-O-(tert-Butoxycarbonyl)-2′-deoxy-2′-fluorouridine-5′-[phenyl-bis(isoamyl-L-aspartyl)]phosphate

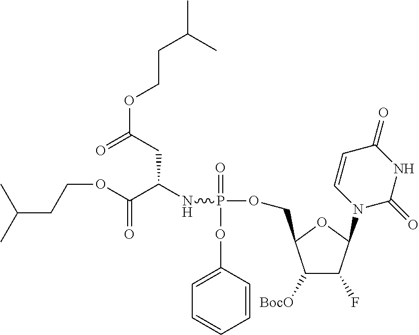

This compound was prepared in 85% yield, using the procedure of example 49.

¹H NMR (300 MHz, DMSO-d₆) δ: 11.50 (s, 1H, NH), 7.86 & 7.67 (m, 1H, Ar—H), 7.36 (m, 2H, Ar—H), 7.19 (m, 3H, Ar—H), 6.16 (m, 1H, NH), 5.94 (m, 1H, OCH), 5.39 (m, 1H, OCH), 5.13 (m, 1H, OCH), 4.10-4.29 (m, 2H, OCH₂), 4.02 (m, 4H, OCH₂), 3.65 (m, 1H, CH), 2.65 (m, 2H, CH₂), 1.62 (m, 2H, CH₂), 1.45 (s, 9H, CH₃), 1.44 (m, 4H, CH₂), 0.86 (m, 12H, CH₃) ppm.

³¹P NMR (202 MHz, DMSO-d₆) δ: 3.72, 3.61 ppm.

### Example 61

### 2′-deoxy-2′-α-fluoro-uridine-5′-[phenyl-bis(isoamyl-L-aspartyl)]phosphate

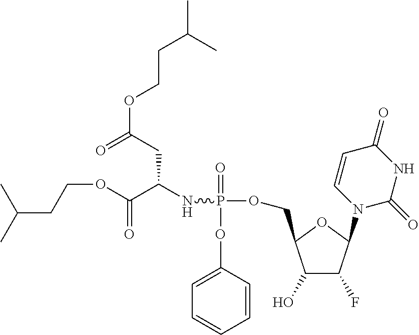

This compound was prepared in 61% yield, according to the procedure of example 54.

¹H NMR (300 MHz, DMSO-d₆) δ: 11.45 (s, 1H, NH), 7.61 (m, 1H, Ar—H), 7.36 (m, 1H, Ar—H), 7.18 (m, 3H, Ar—H), 6.16 (m, 1H, NH), 5.94 (m, 1H, OCH), 5.56 (m, 1H, Ar—H), 4.10-4.30 (m, 10H, OCH & OCH₂), 2.65 (m, 2H, CH₂), 1.62 (m, 2H, CH₂), 1.41 (m, 2H, CH₂), 0.85 (m, 12H, CH₃) ppm.

³¹P NMR (202 MHz, DMSO-d₆) δ: 3.79, 3.60 ppm.

### 7. Synthesis of Glutamate and Serine Phosphoramidate Prodrugs of 2′-C-Me-Uridine

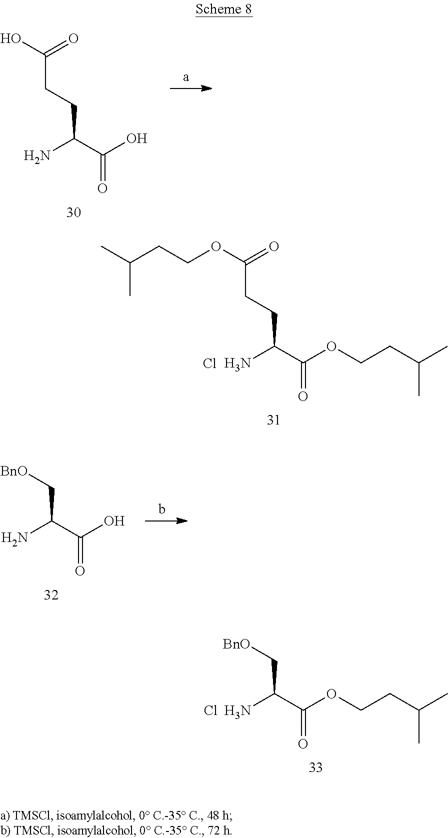 [SMILES: N[C@@H](CCC(=O)O)C(=O)O ; CC(C)CCOC(=O)CC[C@H](N)C(=O)OCCC(C)C ; Cl ; N[C@@H](COCc1ccccc1)C(=O)O ; Cl ; CC(C)CCOC(=O)[C@@H](N)COCc1ccccc1]

### Example 62

### Di-Isoamyl Ester of Glutamic Acid (31)

To a suspension of L-glutamic acid (2.0 g, 13.6 mmol) in anhydrous isoamyl alcohol (60 mL) was added dropwise trimethylchlorosilane (10.4 mL, 81.6 mmol) at 0° C. under argon atmosphere. The mixture was allowed to come to room temperature and stirred for 48 h at 35° C. After evaporation to dryness, hexane was added and the white precipitate was filtered off. Finally, the precipitate was washed several times with hexane to obtain 31 as hydrochloride salt (61%).

¹H NMR (300 MHz, DMSO-d₆): δ=8.71 (br s, 3H, —NH₃⁺), 4.19, 4.06 (5H), 2.52 (2H), 2.08 (2H), 1.67, 1.50 (6H), 0.91 (12H).

¹³C NMR (75 MHz, DMSO-d₆): δ=172.5, 170.0, 65.0, 63.5, 52.0, 37.7, 37.5, 30.0, 26.1, 25.4, 25.2, 23.2, 23.1 ppm

HRMS (ESI+) calcd. for C₁₅H₃₀NO₄ [M+H]⁺ 288.2169. found 288.2166.

### Example 63

### Isoamyl ester of Ser-(OBn)-OH (33)

To a suspension of Ser-(OBn)-OH (1.0 g, 5 mmol) in anhydrous isoamyl alcohol (30 mL) trimethylchlorosilane (4 mL, 30.7 mmol) was added dropwise at 0° C. under argon atmosphere.

The mixture was allowed to come to room temperature and stirred for 72 h at 35° C. After evaporation to dryness, hexane was added and the white precipitate was filtered. Finally the precipitate was washed several times with hexane to obtain 33 as hydrochloride salt (80%).

¹H NMR (300 MHz, DMSO-d₆): δ=8.69 (br s, 3H, —NH₃+), 7.40-7.29 (m, 5H), 4.54 (dd, 2H), 4.34 (t, 1H), 4.25-4.13 (m, 2H), 3.86 (d, 2H), 1.69-1.58 (m, 1H), 1.52-1.43 (m, 2H), 0.88-0.84 (m, 6H);

¹³C NMR (75 MHz, DMSO-d₆): δ=168.7, 138.2, 129.1, 128.6, 128.5, 73.4, 68.3, 65.1, 53.3, 37.5, 25.1, 23.1, 23.0;

HRMS (ESI+) calcd for C₁₅H₂₄NO₃ [M+H]⁺ 266.1751. found 266.1748.

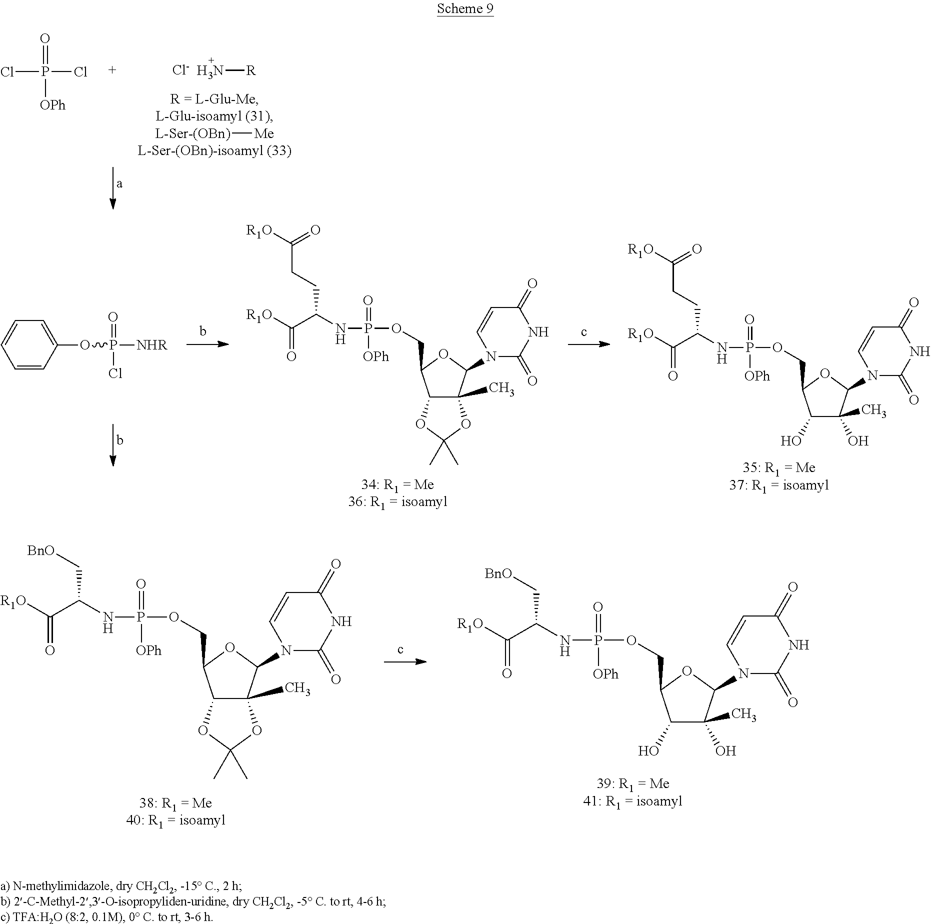

### Example 64

### 2′-C-Methyl-2′,3′-O-isopropyliden-uridine-5′-[phenylbis(methoxy-L-glutamyl)]phosphate (34)

This compound was made according to the procedure for example 14.

Yield: 15%; R_{f}=0.39 (Hexane/EtOAc, 2:8); ³¹P NMR (121 MHz, CDCl₃): δ=3.01, 2.96; HRMS (ESI−) calcd for C₂₆H₃₃N₃O₁₂P [M−H]⁻ 610.1807. found 610.1806.

### Example 65

### 2′-C-Methyl-uridine-5′-[phenylbis(methoxy-L-glutamyl)]phosphate (35)

This compound was made according to the procedure for example 26.

Yield: 77%; R_{f}=0.28 (CH₂Cl₂/MeOH, 9.5:0.5);

¹H NMR (500 MHz, MeOD): δ=7.68-7.66 (2 d, 1H, H-6), 7.39-7.18 (a series of multiplets, 5H, OPh), 5.97, 5.96 (2 s, 1H, H-1′), 5.65-5.61 (2 d, 1H, H-5), 4.56-4.35 (m, 2H, H-5′ & H-5″), 4.11-4.08 (m, 1H, H-4′), 4.00-3.93 (m, 1H, H-α-Glu), 3.80-3.77 (1H, H-3′), 3.69, 3.66, 3.62 (4 s, 6H, OMe), 2.44-2.26 (m, 2H, H-β-Glu), 2.12-1.81 (m, 2H, H-γ-Glu), 1.16, 1.13 (2 s, 3H, —CH₃-2′);

¹³C NMR (125 MHz, MeOD): δ=175.5, 175.2 (—CO-α & —CO-β), 166.7 (C-4), 153.2, 153.1, 153.0 (C-2 & phenyl C), 142.8, 142.7 (C-6), 131.8, 131.7 (phenyl C), 127.2, 127.1 (phenyl C), 122.2-122.1 (phenyl C), 103.7 (C-5), 94.4, 94.3 (C-1′), 82.5, 82.4 (C-4′), 80.5 (C-2′), 74.8, 74.6 (C-3′), 67.3 (d, ²J_{CP}=5.8 Hz, C-5′), 66.8 (d, ²J_{CP}=4.9 Hz, C-5′), 56.2, 56.0 (C-α-Glu), 53.7, 53.0 (OMe), 31.5, 31.4 (C-γ-Glu), 30.8-30.6 (C-β-Glu), 21.0 (CH₃-2′);

³¹P NMR (202 MHz, MeOD): δ=3.95;

HRMS (ESI−) calcd for C₂₃H₂₉N₃O₁₂P [M−H]⁻ 570.1494. found 570.1505.

### Example 66

### 2′-C-Methyl-2′,3′-O-isopropyliden-uridine-5′-[phenylbis(isoamyl-L-glutamyl)]phosphate (36)

This compound was made according to the procedure for example 14.

Yield: 75%; R_{f}=0.59 (CH₂Cl₂/MeOH, 9.5:0.5);

³¹P NMR (121 MHz, CDCl₃): δ=3.06, 3.04 ppm;

HRMS (ESI+) calcd for C₃₄H₅₁N₃O₁₂P [M+H]⁺ 724.3205. found 724.3226.

### Example 67

### 2′-C-Methyl-uridine-5′-[phenylbis(isoamyl-L-glutamyl)]phosphate (37)

This compound was made according to the procedure for example 26.

Yield: 66%; R_{f}=0.39 (CH₂Cl₂/MeOH, 9.5:0.5);

¹H NMR (500 MHz, MeOD): δ=7.68-7.66 (2 d, 1H, H-6), 7.38-7.18 (a series of multiplets, 5H, OPh), 5.97, 5.96 (2 s, 1H, H-1′), 5.66-5.63 (2 d, 1H, H-5), 4.58-4.36 (m, 2H, H-5′ & H-5″), 4.18-4.02 (m, 5H, H-4′ & —OCH₂CH₂CH(CH₃)₂), 4.00-3.92 (m, 1H, H-α-Glu), 3.80-3.76 (d, 1H, H-3′), 2.45-2.26 (m, 2H, H-γ-Glu), 2.09-1.81 (m, 2H, H-β-Glu), 1.70-1.61 (m, 2H, —OCH₂CH₂CH(CH₃)₂), 1.53-1.45 (m, 4H, —OCH₂CH₂CH(CH₃)₂), 1.16, 1.13 (2 s, 3H, —CH₃-2′), 0.92-0.90 (m, 12H, —OCH₂CH₂CH(CH₃)₂);

¹³C NMR (125 MHz, MeOD): δ=175.1, 175.0, 174.9, 174.8 (—CO-α & —CO-β), 166.6 (C-4), 153.1, 153.0, 152.9 (C-2 & phenyl C), 142.7, 142.6 (C-6), 131.8, 131.7 (phenyl C), 127.2 (phenyl C), 122.2-122.1 (phenyl C), 103.7 (C-5), 94.4, 94.2 (C-1′), 82.4, 82.3 (C-4′), 80.5, 80.4 (C-2′), 74.8, 74.5 (C-3′), 67.3 (d, ²J_{CP}=5.2 Hz, C-5′), 66.7 (d, ²J_{CP}=5.2 Hz, C-5′), 65.9, 65.2, 65.1 (—OCH₂CH₂CH(CH₃)₂), 56.3, 56.1 (C-α-Glu), 39.3-39.2 (—OCH₂CH₂CH(CH₃)₂), 31.8-31.7 (C-γ-Glu), 31.0-30.7 (C-β-Glu), 27.1, 27.0 (—OCH₂CH₂CH(CH₃)₂), 23.7-23.6 (—OCH₂CH₂CH(CH₃)₂), 21.1 (CH₃-2′);

³¹P NMR (202 MHz, MeOD): δ=3.94 and 3.90 ppm;

HRMS (ESI−) calcd for C₃₁H₄₅N₃O₁₂P [M−H]⁻ 682.2746. found 682.2753.

### Example 68

### 2′-C-Methyl-2′,3′-O-isopropyliden-uridine-5′-[phenyl(α-methoxy-β-O-benzyl-L-serine)]phosphate (38)

This compound was made according to the procedure for example 14.

Yield: 74%; R_{f}=0.8 (CH₂Cl₂/MeOH, 9.5:0.5);

³¹P NMR (121 MHz, CDCl₃): δ=3.14, 2.77;

HRMS (ESI+) calcd for C₃₀H₃₇N₃O₁₁P [M+H]⁺ 646.2160. found 646.2170.

### Example 69

### 2′-C-Methyl-uridine-5′-[phenyl(α-methoxy-β-O-benzyl-L-serine)]phosphate (39)

This compound was made according to the procedure for example 26.

Yield: 87%; R_{f}=0.38 (CH₂Cl₂/MeOH, 9.5:0.5);

¹H NMR (500 MHz, MeOD): δ=7.70-7.65 (2 d, 1H, H-6), 7.36-7.17 (a series of multiplets, 10H, OPh & CH₂Ph), 5.97, 5.95 (2 s, 1H, H-1′), 5.65-5.59 (2 d, 1H, H-5), 4.60-4.56 (m, 4H, H-5′, H-5″ & CH₂Ph), 4.17-4.05 (m, 1H, H-4′ & H-α-Ser), 3.81-3.52 (m, 6H, H-3′, H-β-Ser & OCH₃—Ser), 1.13 (s, 3H, —CH₃-2′);

¹³C NMR (125 MHz, MeOD): δ=173.9 (d, ³J_{CP}=4.77 Hz, —CO-α), 173.6 (d, ³J_{CP}=6.12 Hz, —CO-α), 166.7 (C-4), 153.1-152.9 (C-2 & phenyl C), 142.8, 142.7 (C-6), 140.0, 139.9 (CH₂Ph), 131.7-122.2 (phenyl C & CH₂Ph), 103.7 (C-5), 94.3, 94.2 (C-1′), 82.4 (C-4′), 80.5, 80.4 (C-2′), 75.0 (CH₂Ph), 74.8, 74.6 (C-3′), 73.2 (d, ³J_{CP}=5.3 Hz, C-β-Ser), 73.0 (d, ³J_{CP}=6.32 Hz, C-β-Ser), 67.2 (d, ²J_{CP}=5.21 Hz, C-5′), 66.7 (d, ²J_{CP}=4.91 Hz, C-5′), 57.3, 57.1 (C-α-Ser), 53.8 (OCH₃—Ser), 21.0 (CH₃-2′);

³¹P NMR (202 MHz, MeOD): δ=4.14 and 3.91;

HRMS (ESI+) calcd for C₂₇H₃₃N₃O₁₁P [M+H]⁺ 606.1847. found 606.1859.

### Example 70

### 2′-C-Methyl-2′,3′-O-isopropyliden-uridine-5′-[phenyl(α-isoamyl-β-O-benzyl-L-serine)]phosphate (40)

This compound was made according to the procedure for example 14.

Yield: 20%; R_{f}=0.53 (Hexane/EtOAc, 1:9);

³¹P NMR (121 MHz, CDCl₃): δ=3.18, 2.87;

HRMS (ESI+) calcd for C₃₄H₄₅N₃O₁₁P [M+H]⁺ 702.2786. found 702.2770.

### Example 71

### 2′-C-Methyl-uridine-5′-[phenyl(α-isoamyl-β-O-benzyl-L-serine)]phosphate (41)

This compound was made according to the procedure for example 26.

Yield: 80%; R_{f}=0.15 (CH₂Cl₂/MeOH, 9.7:0.3);

¹H NMR (500 MHz, MeOD): δ=7.70-7.65 (2 d, 1H, H-6), 7.36-7.17 (a series of multiplets, 10H, OPh & CH₂Ph), 5.98, 5.95 (2 s, 1H, H-1′), 5.65-5.60 (2 d, 1H, H-5), 4.61-4.34 (m, 4H, H-5′, H-5″ & CH₂Ph), 4.21-4.04 (m, 4H, H-4′, —OCH₂CH₂CH(CH₃)₂& H-α-Ser), 3.81-3.52 (m, 3H, H-3′ & H-β-Ser), 1.68-1.59 (m, 1H, —OCH₂CH₂CH(CH₃)₂), 1.51-1.44 (m, 2H, —OCH₂CH₂CH(CH₃)₂), 1.13 (s, 3H, —CH₃-2′), 0.88-0.86 (—OCH₂CH₂CH(CH₃)₂);

¹³C NMR (125 MHz, MeOD): δ=173.5 (d, ³J_{CP}=4.96 Hz, —CO-α), 173.2 (d, ³J_{CP}=6.34 Hz, —CO-α), 166.6 (C-4), 153.1-152.9 (C-2 & phenyl C), 142.8, 142.6 (C-6), 140.0, 139.9 (CH₂Ph), 131.8-122.2 (phenyl C & CH₂Ph), 103.8, 103.7 (C-5), 94.3, 94.2 (C-1′), 82.4 (C-4′), 80.5, 80.4 (C-2′), 75.1 (CH₂Ph), 74.8, 74.6 (C-3′), 73.3 (d, ³J_{CP}=5.40 Hz, C-β-Ser), 73.2 (d, ³J_{CP}=6.48 Hz, C-β-Ser), 67.3 (d, ²J_{CP}=4.83 Hz, C-5′), 66.7 (d, ²J_{CP}=4.83 Hz, C-5′), 66.0 (—OCH₂CH₂CH(CH₃)₂), 57.3, 57.1 (C-α-Ser), 39.3, 39.2 (—OCH₂CH₂CH(CH₃)₂), 26.9 (—OCH₂CH₂CH(CH₃)₂), 23.6 (—OCH₂CH₂CH(CH₃)₂), 21.0 (CH₃-2′);

³¹P NMR (202 MHz, MeOD): δ=4.15 and 3.88;

HRMS (ESI+) calcd for C₃₁H₄₁N₃O₁₁P [M+H]⁺ 662.2473. found 662.2488.

### 8. Synthesis of a Phosporamidate Prodrug of 2′-Deoxy-2′-α-Chloro-Uridine

### Example 72

### Synthesis of 2′-deoxy-2′-chlorouridine

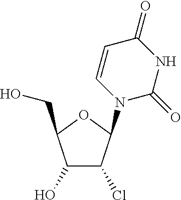

A suspension of O-2, 2′-cyclouridine (1.13 g, 5 mmol) in 1.25 N HCl in isopropanol (10 ml) was stirred at room temperature for 3 hours. The mixture was diluted with dichloromethane (20 ml) and was filtered off. The resulted white solid was suspended in of 1,4-dioxane (180 ml) and heated at 80° C. until a clear solution was obtained. After concentration under reduced pressure, the residue was suspended in 50 ml, filtered off and dried with fresh air to give the title compound as white solid (1.1 g, 84%).

¹H NMR (300 MHz, DMSO-d₆) δ: 11.43 (s, 1H, NH), 7.94 (d, J=8.07 Hz, 1H, Ar—H), 6.02 (d, J=4.4 Hz, 1H), 5.69 (d, J=8.07 Hz, 1H, Ar—H), 5.25 (s, 1H), 4.57 (m, 1H), 4.21 (m, 1H), 3.96 (m, 1H), 3.64 (m, 2H) ppm.

¹³C NMR (75 MHz, DMSO-d₆) δ: 163.12, 150.72, 139.98, 102.25, 88.03, 85.15, 69.28, 62.02, 60.25 ppm.

### Example 73

### 2′-Deoxy-2′-chlorouridine-5′-[phenyl-bis(isoamyl-L-aspartyl)]phosphate

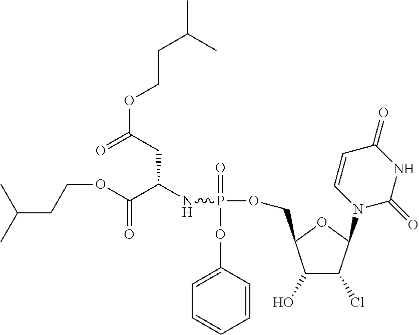

This compound was prepared in 59% yield using the procedure of example 49.

¹H NMR (300 MHz, DMSO-d₆) δ: 11.49 (s, 1H, NH), 7.60 (m, 1H, Ar—H), 7.38 (m, 2H, Ar—H), 7.21 (m, 3H, Ar—H), 6.20 (m, 1H, NH), 6.05 (m, 2H), 5.61 (m, 1H, Ar—H), 4.48 (m, 1H), 4.00-4.30 (m, 8H), 2.66 (m, 2H, CH₂), 1.60 (m, 2H, CH), 1.43 (m, 4H, CH₂), 0.85 (m, 12H, CH₃) ppm.

³¹P NMR (202 MHz, DMSO-d₆) δ: 3.89, 3.76 ppm.

### 9. Biological Evaluation

The cell line ET (luc-ubi-neo/ET) is used, which is a Huh7 human hepatoma cell line that contains an HCV1b/Con1 replicon with a stable luciferase (Luc) reporter and three cell culture-adaptive mutations. The Luc reporter is used as an indirect measure of HCV replication. The activity of the Luc reporter is directly proportional to HCV RNA levels and positive control antiviral compounds behave comparably using either Luc or RNA endpoints. The HCV replicon antiviral evaluation assay examines the effects of compounds at six half-log concentrations each. Human interferon alpha-2b is included in each run as a positive control compound. Sub-confluent cultures of the ET line are plated out into 96-well plates that are dedicated for the analysis of cell numbers (cytotoxicity) or antiviral activity and the next day drugs are added to the appropriate wells. Cells are processed 72 hr later when the cells are still sub-confluent. 6 half-log serial dilutions of the compound has been performed, and derive EC₅₀ values (which is the concentration inhibiting HCV replicon by 50%) and CC₅₀ (concentration decreasing cell viability by 50%). These numbers allows to calculate SI indexes (selectivity index: CC₅₀/EC₅₀) values. HCV replicon levels are assessed as HCV replicon-derived Luc activity. The toxic concentration of drug that reduces cell numbers assessed by the CytoTox-1 cell proliferation assay (Promega) is a colorimetric assay of cell numbers (and cytotoxicity).

Table 1 summarizes the HCV replicon activity of the nucleoside phosphoramidate analogues.

TABLE 1

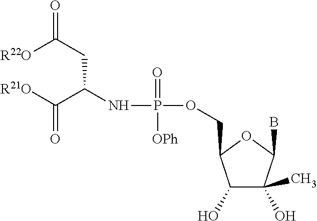

| Cmpd # | B | R²¹ | R²² | EC₅₀ (μM) | CC₅₀ (μM) |
|---|---|---|---|---|---|
| 4 | Cytosine | — | — | 1.34 | >100 |
| 5 | Uracil | — | — | 6.31 | >100 |
| 16a | Cytosine | Me | Me | 3.71 | >100 |
| 16b | Cytosine | Me | Bn | 1.32 | >100 |
| 16c | Cytosine | iPro | iPro | 0.96 | >100 |
| 16d | Cytosine | nBu | nBu | 0.26 | 30.9 |
| 16e | Cytosine | Amyl | Amyl | 0.050 | 9.53 |
| 16f | Cytosine | isoamyl | isoamyl | 0.050 | 9.54 |
| 17a | Uracil | Me | Me | 1.13 | >100 |
| 17b | Uracil | Me | Bn | 0.26 | 43.4 |
| 17c | Uracil | iPro | iPro | 0.29 | 35.7 |
| 17d | Uracil | nBu | nBu | 0.040 | 4.64 |
| 17e | Uracil | Amyl | Amyl | 0.030 | 7.11 |
| 17f | Uracil | isoamyl | isoamyl | 0.030 | 10.0 |

Table 2 summarizes the HCV replicon activity of the 2′-F-nucleoside phosphoramidate analogues.

TABLE 2

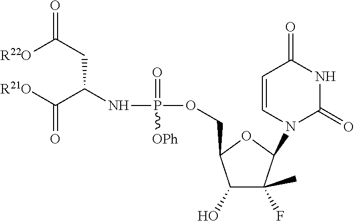

| Cmpd # | R²¹ | R²² | EC₅₀ (μM) | CC₅₀ (μM) |
|---|---|---|---|---|
| 22 | — | — | >100 | >100 |
| 23a | Isoamyl | Isoamyl | 0.06 | 27.7 |
| Mixture | Isoamyl | Isoamyl | 0.06 | 33.1 |
| 23a/23b | | | | |

1. A compound of formula I: 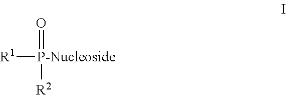
wherein
Nucleoside is a natural nucleoside or a nucleoside analogue;
R¹ has the general formula II: 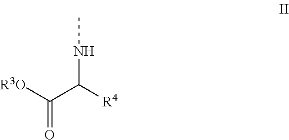
wherein
R³ is selected from the group consisting of aryl, heteroaryl, C₁-C₁₀ alkyl, C₃-C₈-cycloalkyl, C₃-C₈cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
R⁴ is selected from the group consisting of X—COR⁵, and X—O—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of C₁-C₇ alkoxy, aryloxy, C₃-C₁₀ cycloalkoxy, and arylalkyloxy;
R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, and aryl; wherein the aryl moiety of each of said arylalkenyl, aryloxyalkyl, arylalkyl and aryl radicals is optionally substituted with one or more substituents independently selected from the group consisting of halogen, C₁-C₇ alkyl, C₂-C₇ alkenyl, C₂-C₇ alkynyl, halo C₁-C₇ alkyl, nitro, hydroxyl, sulfhydryl, amino, C₁-C₇ alkoxy, C₃-C₁₀ cycloalkoxy, thio C₁-C₇ alkyl, thio C₃-C₁₀ cycloalkyl, thioaryl, cyano, carboxylic acid or esters or amides thereof, alkylamino, cycloalkylamino, alkenylamino, cyclo-alkenylamino, alkynylamino, arylamino, and arylalkylamino;
R² is Y—Ar
wherein Y is O; and Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
wherein when R³ is C₁-C₁₀ alkyl and R⁵ comprises an alkoxy moiety, R⁵ comprises at least 3 carbon atoms;
and/or a pharmaceutical acceptable addition salt thereof and/or a stereoisomer thereof and/or a solvate thereof and/or prodrugs thereof.

2. The compound according to claim 1, wherein the nucleoside is selected from the group consisting of 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, 2′deoxy-2′-α-fluoro-guanosine, gemcitabine, 2′deoxy-2′-α-fluoro-uridine and 2′deoxy-2′-α-chloro-uridine.

3. The compound according to claim 1, wherein the nucleoside is selected from the group consisting of 2′-β-C-Me-Cytidine, 2′-β-C-Me-Uridine, 2′-deoxy-2′-α-fluoro-2′-β-C-methyluridine, 2′-deoxy-2′-α-fluoro-2′-β-C-methylcytidine, and gemcitabine.

4. The compound according to claim 1, having formula IA, 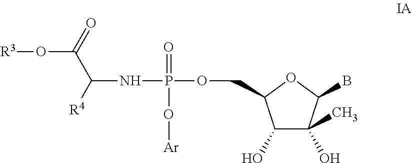
wherein
B is a purine or a pyrimidine base;
Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;
R⁴ is selected from the group consisting of X—COR⁵, and X—O—R⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R⁵ is selected from the group consisting of C₁-C₇ alkoxy, aryloxy, C₃-C₁₀ cycloalkoxy, and arylalkyloxy; and
R⁶ is selected from the group consisting of acyl, alkoxyalkyl, C₃-C₁₀ cycloalkyl-alkyl, C₃₋₁₀ cycloalkyl, heterocyclic-substituted alkyl, acyl-substituted alkyl, carboxylato-substituted alkyl, heterocyclic, halo C₁-C₇ alkyl, C₂-C₇ alkenyl, C₂-C₇ alkynyl, arylalkenyl, aryloxyalkyl, arylalkyl, and aryl.

5. The compound according to claim 1, having formula IB 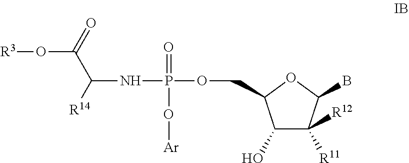
wherein
R¹¹ is OH or halogen, and
when R¹¹ is OH, R¹² is selected from the group consisting of C₁₋₁₀ alkyl, C₂₋₁₀ alkenyl, and C₂₋₁₀ alkynyl;
when R¹¹ is a halogen, R¹² is selected from the group consisting of H, halogen, C₁₋₁₀ alkyl, C₂₋₁₀ alkenyl, and C₂₋₁₀ alkynyl;
B is a purine or a pyrimidine base;
Ar is a monocyclic aryl moiety or a fused bicyclic aryl moiety, either of which aryl moieties is carbocyclic or heterocyclic and is optionally substituted with a halogen, C₁-C₆ alkyl, C₁-C₆ alkoxy;
R³ is selected from the group consisting of C₁-C₁₀ alkyl, aryl(C₁-C₆)alkyl, aryl, heteroaryl, C₃-C₈-cycloalkyl, C₃-C₈cycloalkyl-alkyl, C₂-C₁₀ alkenyl, C₂-C₁₀ alkynyl, hydroxyl C₁-C₁₀ alkyl, halo C₁-C₁₀ alkyl, and alkoxyalkyl;
R¹⁴ is selected from the group consisting of X—COR¹⁵, and X—O—R¹⁶, wherein
X is aryl, heteroaryl, C₁-C₁₀ alkyl, or C₃-C₈-cycloalkyl, and wherein said aryl, heteroaryl, C₁-C₁₀ alkyl, and C₃-C₈-cycloalkyl optionally contains one or more functions, atoms or radicals independently selected from the group consisting of halogen, carbonyl, thiocarbonyl, hydroxyl, thiol, ether, thio-ether, acetal, thio-acetal, amino, imino, oximino, alkyloximino, aminoacid, cyano, acylamino, thioacylamino, carbamoyl, thiocarbamoyl, ureido, thio-ureido, carboxylic acid ester or halide or anhydride or amide, thiocarboxylic acid or ester or thioester or halide or anhydride or amide, nitro, thio C₁₋₇ alkyl, thio C₃₋₁₀ cycloalkyl, hydroxylamino, mercaptoamino, alkyl-amino, cycloalkylamino, alkenylamino, cycloalkenylamino, alkynylamino, arylamino, arylalkylamino, hydroxyalkylamino, mercaptoalkylamino, heterocyclic-substituted alkylamino, hetero-cyclic amino, heterocyclic-substituted arylamino, hydrazine, alkylhydrazino, phenylhydrazino, sulfonyl, sulfinyl and sulfonamido;
R¹⁵ is R¹⁷—O—, wherein R¹⁷ is selected from the group consisting of C₁-C₇ alkyl, aryl, C₃-C₁₀ cycloalkyl, and arylalkyl; and
R¹⁶ is selected from the group consisting of alkoxyalkyl, C₃-C₁₀ cycloalkyl-alkyl, C₃₋₁₀ cycloalkyl, halo C₁-C₇ alkyl, C₂-C₇ alkenyl, C₂-C₇ alkynyl, arylalkyl, and aryl.

6. The compound according to claim 1, wherein Ar is Phenyl.

7. The compound according to claim 1, wherein R³ is C₁-C₁₀ alkyl.

8. (canceled)

9. The compound according to claim 1, wherein X is CH₂.

10. The compound according to claim 1, wherein R⁴ is X—COR⁵, wherein X is C₁-C₁₀ alkyl and wherein R⁵ is selected from the group consisting of C₁-C₇ alkoxy, C₃-C₁₀ cycloalkoxy, aryloxy, and arylalkyloxy.

11. (canceled)

12. The compound according to claim 5, wherein R¹⁴ is X—COOR¹⁷.

13. The compound according to claim 12, wherein R¹⁷ is C₅ alkyl.

14. The compound according to claim 5, wherein R¹¹ is OH and R¹² is CH₃.

15. The compound according to claim 5, wherein R¹¹ is F and R¹² is CH₃.

16. The compound according to claim 5, wherein R¹¹ is F and R¹² is H.

17. The compound according to claim 5, wherein R¹¹ is Cl and R¹² is H.

18. The compound according to claim 5, wherein R¹¹ is Cl and R¹² is CH₃.

19. The compound according to claim 5, wherein R¹¹ and R¹² are both F.

20. The compound according to claim 5, wherein R¹¹ and R¹² are both Cl.

21-26. (canceled)

27. A pharmaceutical composition comprising a therapeutically effective amount of a compound according to claim 1 and one or more pharmaceutically acceptable excipients.

28. A method of prevention or treatment of a viral infection in an animal, mammal or human, comprising the administration of a therapeutically effective amount of a compound according to claim 1, optionally in combination with one or more pharmaceutically acceptable excipients.
